SEQUENCE LISTING (2 sequences)

SEQ ID NO: 1 - protein, 213 aa, synthetic construct
DIQMTQSPSSLSASVGDRVTITCSASSSVSYMNWYQQTPGKAPKRWIYDTSKLASGVPSRFSGSGSGTDYTFTISSLQPEDIATYYCQQWSSNPFTFGQGTKLQITRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, synthetic construct
QVQLVQSGGGVVQPGRSLRLSCKASGYTFTRYTMHWVRQAPGKGLEWIGYINPSRGYTNYNQKVKDRFTISRDNSKNTAFLQMDSLRPEDTGVYFCARYYDDHYCLDYWGQGTPVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2025/0306028 A1
(43) Pub. Date: Oct. 2, 2025

(54) Methods for Prognosing Type 1 Diabetes Treatments

(71) Applicants: Emily K. SIMS, Indianapolis, IN (US); Jay M. SOSENKO, Miami, FL (US); David CUTHBERTSON, Tampa, FL (US); Provention Bio, Inc., Red Bank, NJ (US)

(72) Inventors: Francisco LEON, Bethesda, MD (US); Emily K. SIMS, Indianapolis, IN (US); Jay M. SOSENKO, Miami, FL (US); David CUTHBERTSON, Tampa, FL (US)

(73) Assignee: Provention Bio, Inc., Red Bank, NJ (US)

(21) Appl. No.: 18/693,879

(22) PCT Filed: Sep. 20, 2022
(86) PCT No.: PCT/US2022/076702

§ 371 Date: Mar. 20, 2024

Related U.S. Application Data
(60) Provisional application No. 63/246,184, filed on Sep. 20, 2021.

(51) Int. Cl. G01N 33/66; A61K 39/00; C07K 16/28; G01N 33/68

(52) U.S. Cl. CPC G01N 33/66; C07K 16/2809; G01N 33/6893; A61K 2039/505; C07K 2317/24; G01N 2800/042; G01N 2800/52

(57) ABSTRACT

Type 1 diabetes (T 1 D) is caused by the autoimmune destruction of insulin producing beta cells in the islets of Langerhans leading to dependence on exogeneous insulin injections for survival. A need exists for a treatment that would prevent or delay the onset of clinical T1 D in high-risk individuals. One promising therapy is the anti-CD3 monoclonal antibody teplizumab, as several studies have shown that short-term treatment reduces loss of beta cell function durably, with an observable effect seen as long as 7 years after diagnosis and treatment. Furthermore, improved methods for prognosing such prevention or delay are also needed. Provided herein, in one aspect, is a method of prognosing responsiveness of an anti-CD3 antibody in preventing or delaying the onset of T1 D.

### CROSS REFERENCE TO RELATED APPLICATIONS

This application is a national stage application under 35 U.S.C. § 371 of International Patent Application No. PCT/US2022/076702, filed Sep. 20, 2022, which claims priority to and the benefit of U.S. Provisional Application No. 63/246,184, filed Sep. 20, 2021, the entire disclosure of each of which are incorporated herein by reference.

### GOVERNMENT LICENSE RIGHTS

This invention was made with government support under U01 DK127786, R03 DK117253, and R01DK121929 awarded by the National Institute of Diabetes and Digestive and Kidney Diseases. The government has certain rights in the invention.

### SEQUENCE LISTING

This specification includes a sequence listing submitted herewith, which was created on Jul. 26, 2024 and includes the file entitled 122548. US057.xml having the size of 3,414 bytes, the contents of which are incorporated by reference herein.

### FIELD

The present disclosure relates in general to compositions and methods of preventing or delaying the onset of clinical type 1 diabetes (TID) in subjects at risk, and more particularly the prognosis of using anti-CD3 antibodies in such prevention or delay.

### BACKGROUND

Type 1 diabetes (T1D) is caused by the autoimmune destruction of insulin producing beta cells in the islets of Langerhans leading to dependence on exogeneous insulin injections for survival. Approximately 1.6 million Americans have Type 1 diabetes, and after asthma, it remains one of the most common diseases of childhood. Despite improvements in care, most affected individuals with T1D are not able to consistently achieve desired glycemic targets. For individuals with type 1 diabetes, there are persisting concerns for increased risk of both morbidity and mortality. Two recent studies noted loss of 17.7 life-years for children diagnosed before age 10, and 11 and 13 life-years lost for adult-diagnosed Scottish men and women respectively.

In genetically susceptible individuals, T1D progresses through asymptomatic stages prior to overt hyperglycemia, characterized first by the appearance of autoantibodies (Stage 1) and then dysglycemia (Stage 2). In Stage 2, metabolic responses to a glucose load are impaired but other metabolic indices, for example glycosylated hemoglobin, are normal and insulin treatment is not needed. These immunologic and metabolic features identify individuals who are at high-risk for development of clinical disease with overt hyperglycemia and requirement for insulin treatment (Stage 3). Several immune interventions have been shown to delay decline in beta cell function when studied in recent-onset clinical T1D. One promising therapy is the FcR non-binding anti-CD3 monoclonal antibody teplizumab, as several studies have shown that short-term treatment reduces loss of B cell function durably, with an observable effect seen as long as 7 years after diagnosis and treatment. The drug modifies the function of CD8+ T lymphocytes, which are thought to be important effector cells that cause beta cell killing.

To date, no intervention initiated before the clinical diagnosis (i.e., at Stage 1 or 2) has altered progression to clinical, Stage 3 T1D. Thus, a need exists for a treatment that would prevent or delay the onset of clinical T1D in high-risk individuals. Furthermore, improved methods for prognosing such prevention or delay are also needed.

### SUMMARY

In one aspect, provided herein is a method of prognosing responsiveness of a therapeutic or prophylactic agent for treating type 1 diabetes (T1D), comprising administering a therapeutic or prophylactic agent to a subject in need thereof; and determining a glucose and C-peptide response curve (GCRC) vector of change by plotting change over a period of time of mean glucose values and mean C-peptide values from oral glucose tolerance tests on a 2-dimensional grid, wherein a directionality of the vector of change towards increasing C-peptide and decreasing glucose is indicative of metabolic improvement.

In some embodiments, the oral glucose tolerance tests comprise 1-hour oral glucose tolerance tests. 2-hour oral glucose tolerance tests. 4-hour oral glucose tolerance tests or combinations thereof.

In some embodiments, the method of prognosing responsiveness of a therapeutic or prophylactic agent for treating type 1 diabetes (T1D), comprises administering a therapeutic or prophylactic agent to a subject in need thereof; and determining a glucose and C-peptide response curve (GCRC) vector of change by plotting change over a period of time of mean glucose values and mean C-peptide values from 1-hour oral glucose tolerance tests on a 2-dimensional grid, wherein a directionality of the vector of change towards increasing C-peptide and decreasing glucose is indicative of metabolic improvement.

In some embodiments, the method of prognosing responsiveness of a therapeutic or prophylactic agent for treating type 1 diabetes (T1D), comprises administering a therapeutic or prophylactic agent to a subject in need thereof; and determining a glucose and C-peptide response curve (GCRC) vector of change by plotting change over a period of time of mean glucose values and mean C-peptide values from 2-hour oral glucose tolerance tests on a 2-dimensional grid, wherein a directionality of the vector of change towards increasing C-peptide and decreasing glucose is indicative of metabolic improvement.

In some embodiments, the method of prognosing responsiveness of a therapeutic or prophylactic agent for treating type 1 diabetes (T1D), comprises administering a therapeutic or prophylactic agent to a subject in need thereof; and determining a glucose and C-peptide response curve (GCRC) vector of change by plotting change over a period of time of mean glucose values and mean C-peptide values from 4-hour oral glucose tolerance tests on a 2-dimensional grid, wherein a directionality of the vector of change towards increasing C-peptide and decreasing glucose is indicative of metabolic improvement.

In some embodiments, the method further comprises calculating Within Quadrant Endpoint (WQE) and Ordinal Directional Endpoint (ODE) from the GCRC. In some embodiments, the Within Quadrant Endpoint (WQE) and Ordinal Directional Endpoint (ODE) is based on the directionality quadrant for vector between baseline and 6-month coordinates. In some embodiments, the Within Quadrant Endpoint (WQE) and Ordinal Directional Endpoint (ODE) is based on percent change of glucose and the percent change of C-peptide.

In some embodiments, the therapeutic or prophylactic agent comprises an immunotherapeutic agent. In some embodiments, the immunotherapeutic agent comprises an anti-CD3 antibody or antigen binding fragment thereof. In some embodiments, the anti-CD3 antibody is teplizumab, otelixizumab or foralumab. In one embodiment, the anti-CD3 antibody is teplizumab.

In some embodiments, the effective amount of the therapeutic or prophylactic agent comprises a 10 to 14 day course of daily subcutaneous (SC) injection or intravenous (IV) infusion or oral administration of an anti-CD3 antibody at 10-1100 micrograms/meter squared (μg/m²). In some embodiments, the effective amount of the therapeutic or prophylactic agent comprises a 10 to 14 day course of the anti-CD3 antibody at a total dose of about 9000 μg/m² to about 14000 μg/m².

In some embodiments, the method comprises administering to the subject in need thereof a 14-day course IV infusion of the anti-CD3 antibody at 51 μg/m², 103 μg/m², 207 μg/m², and 413 μg/m², on days 1-4, respectively, and one dose of 826 μg/m² on each of days 5-14.

In some embodiments, the anti-CD3 antibody is teplizumab, otelixizumab or foralumab. In some embodiments, the anti-CD3 antibody is teplizumab.

In some embodiments, the subject is in stage 1, 2, 3, or 4 of T1D. In some embodiments, the subject is in stage 1 or 2 of T1D, and the method can be used for the prognosis of a prophylactic agent in preventing or delaying the onset of T1D.

A further aspect relates to a method of prognosing responsiveness of an anti-CD3 antibody in preventing or delaying the onset of clinical type 1 diabetes (T1D), comprising administering a prophylactically effective amount of an anti-CD3 antibody to a non-clinically diabetic subject who is at risk for clinical T1D; and determining a glucose and C-peptide response curve (GCRC) vector of change by plotting change over a period of time of mean glucose values and mean C-peptide values from oral glucose tolerance tests on a 2-dimensional grid, wherein a directionality of the vector of change towards increasing C-peptide and decreasing glucose is indicative of metabolic improvement.

In some embodiments, the method further comprises calculating Within Quadrant Endpoint (WQE) and Ordinal Directional Endpoint (ODE) from the GCRC. In some embodiments, the Within Quadrant Endpoint (WQE) and Ordinal Directional Endpoint (ODE) is based on the directionality quadrant for vector between baseline and 6-month coordinates. In some embodiments, the Within Quadrant Endpoint (WQE) and Ordinal Directional Endpoint (ODE) is based on percent change of glucose and the percent change of C-peptide.

In some embodiments, the non-clinically diabetic subject is in stage 1 or stage 2 T1D.

In some embodiments, the oral glucose tolerance tests comprise 1-hour oral glucose tolerance tests. 2-hour oral glucose tolerance tests. 4-hour oral glucose tolerance tests or combination thereof.

In some embodiments, the method of prognosing responsiveness of an anti-CD3 antibody in preventing or delaying the onset of clinical type 1 diabetes (T1D) comprises administering a prophylactically effective amount of an anti-CD3 antibody to a non-clinically diabetic subject who is at risk for clinical T1D; and determining a glucose and C-peptide response curve (GCRC) vector of change by plotting change over a period of time of mean glucose values and mean C-peptide values from 1-hour oral glucose tolerance tests on a 2-dimensional grid, wherein a directionality of the vector of change towards increasing C-peptide and decreasing glucose is indicative of metabolic improvement.

In some embodiments, the method of prognosing responsiveness of an anti-CD3 antibody in preventing or delaying the onset of clinical type 1 diabetes (T1D) comprises administering a prophylactically effective amount of an anti-CD3 antibody to a non-clinically diabetic subject who is at risk for clinical T1D; and determining a glucose and C-peptide response curve (GCRC) vector of change by plotting change over a period of time of mean glucose values and mean C-peptide values from 2-hour oral glucose tolerance tests on a 2-dimensional grid, wherein a directionality of the vector of change towards increasing C-peptide and decreasing glucose is indicative of metabolic improvement.

In some embodiments, the method of prognosing responsiveness of an anti-CD3 antibody in preventing or delaying the onset of clinical type 1 diabetes (T1D) comprises administering a prophylactically effective amount of an anti-CD3 antibody to a non-clinically diabetic subject who is at risk for clinical T1D; and determining a glucose and C-peptide response curve (GCRC) vector of change by plotting change over a period of time of mean glucose values and mean C-peptide values from 4-hour oral glucose tolerance tests on a 2-dimensional grid, wherein a directionality of the vector of change towards increasing C-peptide and decreasing glucose is indicative of metabolic improvement.

In some embodiments, the non-clinically diabetic subject who is at risk for clinical T1D is a relative of a patient with T1D.

In some embodiments, the non-clinically diabetic subject who is at risk for clinical T1D is negative for zinc transporter 8 (ZnT8). In some embodiments, the non-clinically diabetic subject who is at risk for clinical T1D is HLA-DR4+. In some embodiments, the non-clinically diabetic subject who is at risk for clinical T1D is not HLA-DR3+. In some embodiments, the non-clinically diabetic subject who is at risk for clinical T1D is HLA-DR4+ and is not HLA-DR3+. In some embodiments, the non-diabetic subject (1) is negative for zinc transporter 8 (ZnT8), (2) is HLA-DR4+, and/or (3) is not HLA-DR3+.

In some embodiments, the method further comprises determining that the non-clinically diabetic subject who is at risk for clinical T1D is negative for zinc transporter 8 (ZnT8) antibodies. In some embodiments, the method further comprises determining that the non-clinically diabetic subject who is at risk for clinical T1D is HLA-DR4+. In some embodiments, the method further comprises determining that the non-clinically diabetic subject who is at risk for clinical T1D is HLA-DR4+ and is not HLA-DR3+. In some embodiments, the method further comprises determining that the non-clinically diabetic subject who is at risk for clinical T1D is HLA-DR4+ and is not HLA-DR3+. In some embodiments, the method further includes determining that the non-clinically diabetic subject who is at risk for clinical T1D (1) is negative for zinc transporter 8 (ZnT8), (2) is HLA-DR4+, and/or (3) is not HLA-DR3+.

In some embodiments, the non-clinically diabetic subject who is at risk for clinical T1D has 2 or more diabetes-related autoantibodies selected from islet cell antibodies (ICA), insulin autoantibodies (IAA), and antibodies to glutamic acid decarboxylase (GAD), tyrosine phosphatase (IA-2/ICA512) or ZnT8.

In some embodiments, the non-clinically diabetic subject who is at risk for clinical T1D has abnormal glucose tolerance on oral glucose tolerance test (OGTT). In some embodiments, the abnormal glucose tolerance on OGTT is a fasting glucose level of 110-125 mg/dL, or 2 hour plasma of ≥140 and <200 mg/dL, or an intervening glucose value at 30, 60. 90 minutes or 4 hours on OGTT>200 mg/dL.

In some embodiments, the anti-CD3 antibody is teplizumab, otelixizumab or foralumab. In some embodiments, the anti-CD3 antibody is teplizumab. In some embodiments, the prophylactically effective amount of the antibody comprises a 10 to 14 day course of subcutaneous (SC) injection or intravenous (IV) infusion or oral administration of the anti-CD3 antibody at 10-1100 micrograms/meter squared (μg/m²). In some embodiments, the effective amount of the therapeutic or prophylactic agent comprises a 10 to 14 day course of the anti-CD3 antibody at a total dose of about 9000 μg/m² to about 14000 μg/m². In some embodiments, the method comprises administering a 14-day course IV infusion at 51 μg/m², 103 μg/m², 207 μg/m², and 413 μg/m², on days 1-4, respectively, and one dose of 826 μg/m² on each of days 5-14.

In some embodiments, the prophylactically effective amount delays median time to clinical diagnosis of T1D by from about 50% to about 90%. In some embodiments, the prophylactically effective amount delays median time to clinical diagnosis of T1D by at least 12 months, at least 18 months, at least 24 months, at least 36 months, at least 48 months, or at least 60 months.

In some embodiments, the determining of TIGIT+KLRG1+CD8+ T-cells is by flow cytometry.

In some embodiments, the method further includes determining a decrease in a percentage of CD8+ T cells expressing proliferation markers Ki67 and/or CD57.

### BRIEF DESCRIPTION OF THE DRAWINGS

FIGS. 1A-1F: Changes in GCRC vectors show opposite directionality between placebo and teplizumab-treated groups. Individual participant GCRC vectors of change as well as mean treatment group vectors were plotted for the interval from baseline study visit (time of randomization) to 3 months on treatment (A-D) and baseline to 6 months on treatment (E-H). A-B: Changes in individual GCRC vectors from baseline to 3 months on treatment are plotted for placebo (A) and teplizumab-treated groups (B). High-risk values (increasing glucose, decreasing C-peptide) are shown with dashed lines, with low-risk (decreasing glucose, increasing C-peptide) vectors shown with thick lines. Frequency of vector quadrant distribution was significantly different between treatment groups (p=0.045). C-D: Mean GCRCs are plotted at the time of randomization (baseline, shown in solid line), and at the time of the OGTT performed after 3 months on study (shown in dashed line) for placebo-treated and teplizumab-treated groups. Mean centroid values for glucose and C-peptide coordinates are depicted as dots within each GCRC, with vectors of change indicated. The change in GCRC centroid values over this period shows directionality toward the upper left portion of the grid for the placebo group, indicating increasing glucose and decreasing C-peptide values. The teplizumab-treated group shows opposite directionality, towards the lower right portion of the grid, suggesting increasing C-peptide and decreasing glucose. E-F: Changes in individual GCRC vectors from baseline to 6 months on treatment are plotted for placebo (E) and teplizumab-treated groups (F). Frequency of vector quadrant distribution was significantly different between treatment groups (p=0.0044). G-H: Mean GCRCs are plotted at the time of randomization (baseline, shown in solid line), and at the time of the OGTT performed after 6 months on study (shown in dashed line) for placebo-treated and teplizumab-treated groups. Mean centroid values for glucose and C-peptide coordinates are depicted as dots within each GCRC, with vectors of change indicated. The change in GCRC centroid values over this period shows directionality toward the upper left portion of the grid for the placebo group, indicating increasing glucose and decreasing C-peptide values. The teplizumab-treated group shows opposite directionality, towards the lower right portion of the grid, suggesting increasing C-peptide and decreasing glucose. At 3 months: n=29 for placebo: n=41 for teplizumab. At 6 months: n=24 for placebo: n=44 for teplizumab. Absolute frequencies for individual vector directional quadrants for each period are displayed in Table 1.

FIGS. 2A-2B. Statistical Assessments of a Treatment Effect Using Vector Angles within Directional Quadrants. Unadjusted individual values for changes in A, the within quadrant endpoint (WQE) treatment endpoint and B, the ordinal directional endpoint (ODE) are plotted by treatment group for the period from baseline to 3 months and baseline to 6 months. For ODE treatment group comparisons, at 3 months, p=0.018 before and p=0.038 after adjustments for age and BMI. At 6 months p<0.001 before and p=0.002 after adjustment. For WQE treatment group comparisons, at 3 months p=0.026 before and p=0.072 after adjustments for age and BMI. At 6 months, p<0.001 before and after adjustment. * unadjusted p value <0.05; *** unadjusted p value <0.001. At 3 months, n=29 placebo-treated and 41 teplizumab-treated individuals. At 6 months, n=24 placebo-treated and 44 teplizumab-treated individuals.

FIGS. 3A-3C. Glucose C-peptide Response Curves (GCRCs) Allow for Visualization and Quantification of the Evolving Relationship between Glucose and C-peptide as Type 1 Diabetes Develops. A. Hypothetical GCRC's showing typical glucose and C-peptide values at 30) (open circle). 60. 90, and 120 minute (open square) timepoints of an oral glucose tolerance test are plotted for individuals at the time of diagnosis of type 1 diabetes (dashed line) and 6 months before diagnosis. Mean centroid values for glucose and C-peptide coordinates are indicated as dots within each GCRC. Vector showing the change in mean GCRC centroid values over this period shows directionality toward the upper left portion of the grid, indicating increasing glucose and decreasing C-peptide values. B. Conceptual diagram displaying application of GCRC vector to create a right triangle, allowing for combined application of directional quadrant of change and angles generated by the triangle to quantify changes in metabolic function. The calculated angle is the angle between the vector (hypotenuse) and the horizontal side of the right triangle. C. Hypothetical example of vectors for each of the 4 directional quadrants, which emanate from a baseline centroid which has glucose and C-peptide values fixed at ( ) The calculated angles between the horizontal and the vector are also shown.

FIG. 4. Angles Utilized for WQE (qangle, indicated in bold) Based on Directional Quadrant of GCRC Vector of Change: The metabolic changes of glucose and C-peptide over the 6-month period would fall into 1 of the 4 directional quadrants indicated. Each quadrant includes calculated vector angles between the vector and the horizontal border as well as the vector and the vertical border. For vectors falling within the upper quadrants, the qangle (the angle used to optimize the model for use as an endpoint, indicated in red) was calculated between the vector and the horizontal border. For vectors falling within the lower quadrants, the qangle was calculated between the vector and the vertical border. In this example, for the RUQ, the vector angle of 62° between the vector and the horizontal axis would be used as the qangle for the development of the WQE. However, for the LRQ, the calculated vector angle of 40° (between the vector and the vertical axis) was utilized for the qangle.

### DETAILED DESCRIPTION

Aspects of the disclosure relate to methods prognosing responsiveness of a therapeutic or prophylactic agent for treating type 1 diabetes (T1D). In some embodiments, the method of prognosing responsiveness of a therapeutic or prophylactic agent for treating type 1 diabetes (T1D), comprising: administering a therapeutic or prophylactic agent to a subject in need thereof: constructing glucose and C-peptide response curve (GCRC) by plotting mean glucose and C-peptide values from 1-hour. 2-hour or 4-hour oral glucose tolerance tests on a 2-dimensional grid: visually observing changes in GCRC shape and movement to determine a metabolic improvement and optionally, calculating Within Quadrant Endpoint (WQE) and Ordinal Directional Endpoint (ODE) from the GCRC.

In one aspect, provided herein is a method of prognosing responsiveness of a therapeutic or prophylactic agent for treating type 1 diabetes (T1D), comprising: administering a therapeutic or prophylactic agent to a subject in need thereof; and determining a glucose and C-peptide response curve (GCRC) vector of change by plotting change over a period of time of mean glucose values and mean C-peptide values from oral glucose tolerance tests on a 2-dimensional grid, wherein a directionality of the vector of change towards increasing C-peptide and decreasing glucose is indicative of metabolic improvement.

In some embodiments, the oral glucose tolerance tests comprise 1-hour oral glucose tolerance tests. 2-hour oral glucose tolerance tests. 4-hour oral glucose tolerance tests or combinations thereof.

In some embodiments, the method of prognosing responsiveness of a therapeutic or prophylactic agent for treating type 1 diabetes (T1D), comprises administering a therapeutic or prophylactic agent to a subject in need thereof; and determining a glucose and C-peptide response curve (GCRC) vector of change by plotting change over a period of time of mean glucose values and mean C-peptide values from 1-hour oral glucose tolerance tests on a 2-dimensional grid, wherein a directionality of the vector of change towards increasing C-peptide and decreasing glucose is indicative of metabolic improvement.

In some embodiments, the method of prognosing responsiveness of a therapeutic or prophylactic agent for treating type 1 diabetes (T1D), comprises administering a therapeutic or prophylactic agent to a subject in need thereof; and determining a glucose and C-peptide response curve (GCRC) vector of change by plotting change over a period of time of mean glucose values and mean C-peptide values from 2-hour oral glucose tolerance tests on a 2-dimensional grid, wherein a directionality of the vector of change towards increasing C-peptide and decreasing glucose is indicative of metabolic improvement.

In some embodiments, the method of prognosing responsiveness of a therapeutic or prophylactic agent for treating type 1 diabetes (T1D), comprises administering a therapeutic or prophylactic agent to a subject in need thereof; and determining a glucose and C-peptide response curve (GCRC) vector of change by plotting change over a period of time of mean glucose values and mean C-peptide values from 4-hour oral glucose tolerance tests on a 2-dimensional grid, wherein a directionality of the vector of change towards increasing C-peptide and decreasing glucose is indicative of metabolic improvement.

In some embodiments, the step of determining the GCRC vectors of change comprises determining mean glucose values and mean C-peptide values after administration of the therapeutic or prophylactic agent, and determining mean baseline glucose values and mean baseline C-peptide values prior to administration of the therapeutic or prophylactic agent.

In some embodiments, the method comprises administering therapeutic or prophylactic agent to a plurality of subjects in need thereof and determining a plurality of GCRC vectors of change. In some embodiments, a frequency of directionality of the plurality of the vectors of change towards increasing C-peptide and decreasing glucose is indicative of metabolic improvement.

In some embodiments, the method further comprises plotting mean baseline GCRC from the plurality of subjects and mean treatment GCRC from the plurality of subjects, determining mean baseline centroid values for glucose and C-peptide coordinates for the baseline GCRC and mean treatment centroid values for glucose and C-peptide coordinates for the treatment GCRC, and determining a vector of change between the mean baseline centroid value and the mean treatment centroid value. In some embodiments, the change in GCRC centroid values over a period of time and/or the change of directionality of the vector of change towards increasing C-peptide and decreasing glucose is indicative of metabolic improvement. In some embodiments, the period of time comprises 3 months. 4 months. 5 months. 6 months after administration of the therapeutic or prophylactic agent.

In some embodiments, the method comprises (a) administering a placebo to a first plurality of subject in need thereof, determining a first plurality of GCRC vectors of change, determining a first frequency of directionality of the plurality of the vectors of change towards increasing C-peptide and decreasing glucose. (b) administering a therapeutic or prophylactic agent to a second plurality of subject in need thereof, determining a second plurality of GCRC vectors of change, determining a second frequency of directionality of the plurality of the vectors of change towards increasing C-peptide and decreasing glucose, wherein a second frequency that is significantly higher than the first frequency is indicative of metabolic improvement.

In some embodiments, the method further comprises calculating Within Quadrant Endpoint (WQE) and Ordinal Directional Endpoint (ODE) from the GCRC. In some embodiments, the Within Quadrant Endpoint (WQE) and Ordinal Directional Endpoint (ODE) is based on the directionality quadrant for vector between baseline and 6-month coordinates. In some embodiments, the Within Quadrant Endpoint (WQE) and Ordinal Directional Endpoint (ODE) is based on percent change of glucose and the percent change of C-peptide.

Note that the method can be used for the prognosis of any therapeutic or prophylactic agent in treating or preventing any stage of T1D. T1D is characterized by destruction of most insulin-producing beta cells by an autoimmune response. Patients with established T1D have residual beta cells, but these do not thrive due to the autoimmune disease, which destroys them upon proliferation. There are 4 stages of T1D: stage 1—multiple (at least 2) islet antibodies, normal blood glucose, pre-symptomatic: stage 2—multiple islet antibodies, raised blood glucose, pre-symptomatic: stage 3—islet autoimmunity, raised blood glucose, symptomatic: stage 4—long standing type 1 diabetes. In some aspects of the disclosure, the method results in regeneration of beta cells.

Provided herein, in some embodiments, is a method of prognosing responsiveness of an anti-CD3 antibody in preventing or delaying the onset of clinical type 1 diabetes (T1D), comprising administering a prophylactically effective amount of an anti-CD3 antibody to a non-clinically diabetic subject who is at risk for clinical T1D; and determining a glucose and C-peptide response curve (GCRC) vector of change by plotting change over a period of time of mean glucose values and mean C-peptide values from oral glucose tolerance tests on a 2-dimensional grid, wherein a directionality of the vector of change towards increasing C-peptide and decreasing glucose is indicative of metabolic improvement.

In some embodiments, the oral glucose tolerance tests comprise 1-hour oral glucose tolerance tests. 2-hour oral glucose tolerance tests. 4-hour oral glucose tolerance tests or combination thereof.

In some embodiments, the method of prognosing responsiveness of an anti-CD3 antibody in preventing or delaying the onset of clinical type 1 diabetes (T1D) comprises administering a prophylactically effective amount of an anti-CD3 antibody to a non-clinically diabetic subject who is at risk for clinical T1D; and determining a glucose and C-peptide response curve (GCRC) vector of change by plotting change over a period of time of mean glucose values and mean C-peptide values from 1-hour oral glucose tolerance tests on a 2-dimensional grid, wherein a directionality of the vector of change towards increasing C-peptide and decreasing glucose is indicative of metabolic improvement.

In some embodiments, the method of prognosing responsiveness of an anti-CD3 antibody in preventing or delaying the onset of clinical type 1 diabetes (T1D) comprises administering a prophylactically effective amount of an anti-CD3 antibody to a non-clinically diabetic subject who is at risk for clinical T1D; and determining a glucose and C-peptide response curve (GCRC) vector of change by plotting change over a period of time of mean glucose values and mean C-peptide values from 2-hour oral glucose tolerance tests on a 2-dimensional grid, wherein a directionality of the vector of change towards increasing C-peptide and decreasing glucose is indicative of metabolic improvement.

In some embodiments, the method of prognosing responsiveness of an anti-CD3 antibody in preventing or delaying the onset of clinical type 1 diabetes (T1D) comprises administering a prophylactically effective amount of an anti-CD3 antibody to a non-clinically diabetic subject who is at risk for clinical T1D; and determining a glucose and C-peptide response curve (GCRC) vector of change by plotting change over a period of time of mean glucose values and mean C-peptide values from 4-hour oral glucose tolerance tests on a 2-dimensional grid, wherein a directionality of the vector of change towards increasing C-peptide and decreasing glucose is indicative of metabolic improvement.

In some embodiments, the method further comprises calculating Within Quadrant Endpoint (WQE) and Ordinal Directional Endpoint (ODE) from the GCRC. In some embodiments, the Within Quadrant Endpoint (WQE) and Ordinal Directional Endpoint (ODE) is based on the directionality quadrant for vector between baseline and 6-month coordinates. In some embodiments, the Within Quadrant Endpoint (WQE) and Ordinal Directional Endpoint (ODE) is based on percent change of glucose and the percent change of C-peptide.

In some embodiments, the method further comprises determining, prior to or after the administering step, that the non-clinically diabetic subject who is at risk for clinical T1D has more than about 5% to more than about 10% TIGIT+KLRG1+CD8+ T-cells in all CD3+ T cells, which is indicative of successful prevention or delay of the onset of clinical T1D. In some embodiments, the determining of the percentage of TIGIT+KLRG1+CD8+ T-cells by flow cytometry. In some embodiments, the method further comprises determining a decrease in a percentage of CD8+ T cells expressing proliferation markers Ki67 and/or CD57.

In some embodiments, the non-clinically diabetic subject is in stage 1 or stage 2 T1D.

In some embodiments, the method of prognosing responsiveness of an anti-CD3 antibody in preventing or delaying the onset of type 1 diabetes (T1D), comprises: providing a non-clinically diabetic subject who is at risk for T1D: administering a prophylactically effective amount of an anti-CD3 antibody to the non-clinically diabetic subject: constructing glucose and C-peptide response curve (GCRC) by plotting mean glucose and C-peptide values from 1-hour. 2-hour or 4-hour oral glucose tolerance tests on a 2-dimensional grid: visually observing changes in GCRC shape and movement to determine a metabolic improvement; and optionally, calculating Within Quadrant Endpoint (WQE) and Ordinal Directional Endpoint (ODE) from the GCRC.

### Definitions

Certain terms are defined herein below. Additional definitions are provided throughout the application.

As used herein, the articles “a” and “an” refer to one or more than one, e.g., to at least one, of the grammatical object of the article. The use of the words “a” or “an” when used in conjunction with the term “comprising” herein may mean “one.” but it is also consistent with the meaning of “one or more.” “at least one.” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g, scFv); and multispecific antibodies formed from antibody fragments.

As used herein, the term “glucose and C-peptide response curve” refers to the plot of change over a period of time of mean glucose values and mean C-peptide values from 2-hour oral glucose tolerance tests on a 2-dimensional grid.

In some embodiments, GCRC can be generated by plotting mean glucose (y-axis) and C-peptide values (x-axis) from OGTTs (30, 60, 90, 120 minutes and 4 hours) on a 2dgrid.

As used herein, the term “prophylactic agent” refer to CD3 binding molecules such as teplizumab which can be used in the prevention, treatment, management or amelioration of one or more symptoms of T1D.

As used herein, the terms “treat,” “treatment,” and “treating” refer to any indicia of success in the treatment or amelioration of an injury, pathology, condition, or symptom (e.g., cognitive impairment), including any objective or subjective parameter such as abatement: remission: diminishing of symptoms or making the symptom, injury, pathology or condition more tolerable to the patient: reduction in the rate of symptom progression: decreasing the frequency or duration of the symptom or condition: or, in some situations, preventing the onset of the symptom. The treatment or amelioration of symptoms can be based on any objective or subjective parameter: including, e.g., the result of a physical examination.

As used herein, the term “onset” of disease with reference to Type-1 diabetes refers to a patient meeting the criteria established for diagnosis of Type-1 diabetes by the American Diabetes Association (see, Mayfield et al., 2006, Am. Fam. Physician 58:1355-1362).

As used herein, the terms “prevent”. “preventing” and “prevention” refer to the prevention of the onset of one or more symptoms of T1D in a subject resulting from the administration of a prophylactic or therapeutic agent.

As used herein, a “protocol” includes dosing schedules and dosing regimens. The protocols herein are methods of use and include prophylactic and therapeutic protocols. A “dosing regimen” or “course of treatment” may include administration of several doses of a therapeutic or prophylactic agent over 1 to 20 days.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a primate (e.g., a monkey or a human), and more preferably a human.

As used herein, the term “prophylactically effective amount” refers to that amount of teplizumab sufficient to result in the delay or prevention of the development, recurrence or onset of one or more symptoms of T1D. In some embodiments, a prophylactically effective amount preferably refers to the amount of teplizumab that delays a subject's onset of T1D by at least 20%, by at least 25%, by at least 30%, by at least 35%, by at least 40%, by at least 45%, by at least 50%, by at least 55%, by at least 60%, by at least 65%, by at least 70%, by at least 75%, by at least 80%, by at least 85%, by at least 90%, by at least 95%.

Various aspects of the disclosure are described in further detail below. Additional definitions are set out throughout the specification.

### Anti-CD3 Antibodies and Pharmaceutical Compositions

The terms “anti-CD3 antibody” and “an antibody that binds to CD3” refer to an antibody or antibody fragment that is capable of binding cluster of differentiation 3 (CD3) with sufficient affinity such that the antibody is useful as a prophylactic, diagnostic and/or therapeutic agent in targeting CD3. In some embodiments, the extent of binding of an anti-CD3 antibody to an unrelated, non-CD3 protein is less than about 10% of the binding of the antibody to CD3 as measured, e.g., by a radioimmunoassay (RIA). In some embodiments, an antibody that binds to CD3 has a dissociation constant (Kd) of <1 μM, <100 nM, <10 nM, <1 nM, <0.1 nM, <0.01 nM, or <0.001 nM (e.g. 10⁻⁸ M or less, e.g. from 10⁻⁸ M to 10⁻¹³ M, e.g., from 10⁻⁹ M to 10⁻¹³ M). In some embodiments, an anti-CD3 antibody binds to an epitope of CD3 that is conserved among CD3 from different species.

In some embodiments, the anti-CD3 antibody can be ChAglyCD3 (otelixizumab). Otelixizumab is a humanized Fc nonbinding anti-CD3, which was evaluated initially in phase 2 studies by the Belgian Diabetes Registry (BDR) and then developed by Tolerx, which then partnered with GSK to conduct the phase 3 DEFEND new onset TlD trials (NCT00678886, NCT01123083, NCT00763451). Otelixizumab is administered IV with infusions over 8 days. See, e.g., Wiczling et al., J. Clin. Pharmacol. 50 (5) (May 2010) 494-506; Keymeulen et al., N Engl J Med. 2005; 352:2598-608; Keymeulen et al., Diabetologia. 2010; 53:614-23; Hagopian et al., Diabetes. 2013; 62:3901-8; Aronson et al., Diabetes Care. 2014; 37:2746-54; Ambery et al., Diabet Med. 2014; 31:399-402; Bolt et al., Eur. J. Immunol. lYY3. 23: 403-411; Vlasakakis et al., Br J Clin Pharmacol (2019) 85 704-714; Guglielmi et al, Expert Opinion on Biological Therapy, 16:6, 841-846; Keymeulen et al., N Engl J Med 2005; 352:2598-608; Keymeulen et al., BLOOD 2010, VOL 115, No. 6; Sprangers et al., Immunotherapy (2011) 3(11), 1303-1316; Daifotis et al., Clinical Immunology (2013) 149, 268-278; all incorporated herein by reference.

In some embodiments, the anti-CD3 antibody can be visilizumab (also called HuM291; Nuvion). Visilizumab is a humanized anti-CD3 monoclonal antibody characterized by a mutated IgG2 isotype, lack of binding to Fcγ receptors, and the ability to induce apoptosis selectively in activated T cells. It was evaluated in patients in graft-versus-host disease (NCT00720629; NCT00032279) and in ulcerative colitis (NCT00267306) and Crohn's Disease (NCT00267709). See, e.g., Sandborn et al., Gut 59 (11) (November 2010) 1485-1492, incorporated herein by reference.

In some embodiments, the anti-CD3 antibody can be foralumab, a fully human anti-CD3 monoclonal antibody being developed by Tiziana Life Sciences, PLC in NASH and T2D (NCT03291249). See, e.g., Ogura et al., Clin Immunol. 2017; 183:240-246; Ishikawa et al., Diabetes. 2007; 56(8):2103-9; Wu et al J Immunol. 2010; 185(6):3401-7; all incorporated herein by reference. Foralumab is a fully human monoclonal antibody that binds to CD3 epsilon. (see U.S. Pat. No. 10,688,186 incorporated by reference in its entirety.)

### Teplizumab

In some embodiments, the anti-CD3 antibody can be teplizumab. Teplizumab, also known as hOKT3yl(Ala-Ala) (containing an alanine at positions 234 and 235) is an anti-CD3 antibody that had been engineered to alter the function of the T lymphocytes that mediate the destruction of the insulin-producing beta cells of the islets of the pancreas. Teplizumab binds to an epitope of the CD3ε chain expressed on mature T cells and by doing so changes their function. Sequences and compositions of teplizumab are disclosed in U.S. Pat. Nos. 6,491,916; 8,663,634; and 9,056,906, each incorporated herein by reference in its entirety. The full sequences of light and heavy chains are set forth below. Bolded portions are the complementarity determining regions.

Teplizumab Light Chain (SEQ ID NO: 1):
DIQMTQSPSSLSASVGDRVTITCSASSSVSYMNWYQQTPGKAPKRWIY
DTSKLASGVPSRFSGSGSGTDYTFTISSLQPEDIATYYCQQWSSNPFT
FGQGTKLQITRTVAAPSVFIFPPSDEQLKSGTASVVCLLNNFYPREAK
VQWKVDNALQSGNSQESVTEQDSKDSTYSLSSTLTLSKADYEKHKVY
ACEVTHQGLSSPVTKSFNRGEC
Teplizumab Heavy Chain (SEQ ID NO: 2):
QVQLVQSGGGVVQPGRSLRLSCKASGYTFTRYTMHWVRQAPGKGLEWI
GYINPSRGYTNYNQKVKDRFTISRDNSKNTAFLQMDSLRPEDTGVYFC
ARYYDDHYCLDYWGQGTPVTVSSASTKGPSVFPLAPSSKSTSGGTAAL
GCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGP
SVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHE
ALHNHYTQKSLSLSPGK

In some embodiments, provided herein, is a pharmaceutical composition. Such compositions comprise a prophylactically effective amount of an anti-CD3 antibody, and a pharmaceutically acceptable carrier. In some embodiments, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like (See, for example, Handbook of Pharmaceutical Excipients, Arthur H. Kibbe (ed., 2000, which is incorporated by reference herein in its entirety), Am. Pharmaceutical Association, Washington, D.C.

The composition, if desired, can also contain minor amounts of wetting or emulsifying agents, or pH buffering agents. These compositions can take the form of solutions, suspensions, emulsion, tablets, pills, capsules, powders, sustained release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In some embodiments, the pharmaceutical compositions are sterile and in suitable form for administration to a subject, preferably an animal subject, more preferably a mammalian subject, and most preferably a human subject.

In some embodiments, it may be desirable to administer the pharmaceutical compositions locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering the anti-CD3 antibody, care must be taken to use materials to which the anti-CD3 antibody does not absorb.

In some embodiments, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In some embodiments, the composition can be delivered in a controlled release or sustained release system. In some embodiments, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In some embodiments, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In some embodiments, the polymer used in a sustained release formulation is inert, free of leachable impurities, stable on storage, sterile, and biodegradable. In some embodiments, a controlled or sustained release system can be placed in proximity of the therapeutic target, i.e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention or fragments thereof. See, e.g., U.S. Pat. No. 4,526,938; PCT Publication No. WO 91/05548; PCT Publication No. WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854; and Lam et al., 1997, Proc. Int'l. Symp. Control Rel. Bioact. Mater. 24:759-760, each of which is incorporated herein by reference in its entirety.

A pharmaceutical composition can be formulated to be compatible with its intended route of administration. Examples of routes of administration include, but are not limited to, parenteral, e.g., intravenous, intradermal, subcutaneous, oral, intranasal (e.g., inhalation), transdermal (topical), transmucosal, and rectal administration. In some embodiments, the composition is formulated in accordance with routine procedures as a pharmaceutical composition adapted for intravenous, subcutaneous, intramuscular, oral, intranasal or topical administration to human beings. In some embodiments, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

The compositions may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

In some embodiments, the disclosure provides dosage forms that permit administration of the anti-CD3 antibody continuously over a period of hours or days (e.g., associated with a pump or other device for such delivery), for example, over a period of 1 hour, 2 hours, 3 hours, 4 hours, 6 hours, 8 hours, 10 hours, 12 hours, 16 hours, 20 hours, 24 hours, 30 hours, 36 hours, 4 days, 5 days, 7 days, 10 days or 14 days. In some embodiments, the invention provides dosage forms that permit administration of a continuously increasing dose, for example, increasing from 51 ug/m²/day to 826 ug/m²/day over a period of 24 hours, 30 hours, 36 hours, 4 days, 5 days, 7 days, 10 days or 14 days.

The compositions can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of the compositions disclosed herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the disclosure provides that the anti-CD3 antibodies, or pharmaceutical compositions thereof, can be packaged in a hermetically sealed container such as an ampoule or sachette indicating the quantity of the agent. In some embodiments, the anti-CD3 antibody, or pharmaceutical compositions thereof is supplied as a dry sterilized lyophilized powder or water free concentrate in a hermetically sealed container and can be reconstituted, e.g., with water or saline to the appropriate concentration for administration to a subject. Preferably, the anti-CD3 antibody, or pharmaceutical compositions thereof is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic agents, or pharmaceutical compositions herein should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In some embodiments, the pharmaceutical composition is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/ml, at least 25 mg/ml, at least 50 mg/ml, at least 75 mg/ml or at least 100 mg/ml. The liquid form should be stored at between 2° C. and 8° C. in its original container.

In some embodiments, the disclosure provides that the composition of the invention is packaged in a hermetically sealed container such as an ampoule or sachette indicating the quantity of the anti-CD3 antibody.

The compositions may, if desired, be presented in a pack or dispenser device that may contain one or more unit dosage forms containing the active ingredient. The pack may, for example, comprise metal or plastic foil, such as a blister pack.

The amount of the composition of the invention which will be effective in the prevention or amelioration of one or more symptoms associated with TlD can be determined by standard clinical techniques. The precise dose to be employed in the formulation will also depend on the route of administration and the seriousness of the condition, and should be decided according to the judgment of the practitioner and each patient's circumstances. Effective doses may be extrapolated from dose-response curves derived from in vitro or animal model test systems.

### Methods and Use

The method disclosed herein can be used for the prognosis of any therapeutic or prophylactic agent in treating or preventing any stage of TlD, including stage 1, 2, 3, or 4. In some embodiments, the present disclosure encompasses administration of anti-human CD3 antibodies such as teplizumab to individuals predisposed to develop type 1 diabetes or with pre-clinical stages of type 1 diabetes, but who do not meet the diagnosis criteria as established by the American Diabetes Association or the Immunology of Diabetes Society to prevent or delay the onset of type 1 diabetes and/or to prevent or delay the need for administration of exogenous insulin to such patients.

In some embodiments, new metabolic endpoints can be used to detect an agent's effect on treatment or prevention of TlD after administration. Glucose and C-peptide response curves (GCRCs) can be constructed by, e.g., plotting mean glucose and C-peptide values from 2-hour oral glucose tolerance tests on a 2-dimensional grid. Changes in GCRC shape and movement can be compared visually between placebo and treatment groups. While GCRC changes can reflect marked metabolic deterioration in the placebo group, if GCRC changes in the treatment group suggest metabolic improvement, then the agent is effective in treating or preventing TlD. Quantitative comparisons, including two novel metabolic endpoints that indicate GCRC changes, the Within Quadrant Endpoint (WQE) and the Ordinal Directional Endpoint (ODE), can also be used.

In some embodiments, Within Quadrant Endpoint (WQE) can be used. Specifically, the prediction of TlD risk or prognosis can be enhanced by dividing the 3600 continuum into its 4 directional quadrants from 0° to 90°. Each directional quadrant can be considered to have its own characteristic risk that cab be predictive of overall risk when included together in a model. The directional quadrants can be designated as:
Right Upper Quadrant (RUQ)
Left Upper Quadrant (LUQ)
Right Lower Quadrant (RLQ)
Left Lower Quadrant (LLQ)

Angles can be calculated from right triangles formed according to the directionality quadrant of an individual's vector of change. Negative calculated angles can be transformed to positive. The percent change of the centroid glucose from baseline to 6 months on the y-axis and percent change of the centroid C-peptide from baseline to 6 months on the x-axis can be used for the calculation of the angle. The hypotenuse of the triangle represents the distance from the baseline GCRC centroid from baseline to 6 months (i.e., vector for change). The formulas for the calculated angle and hypotenuse are shown below:

$$radian = arc tangent ( \% change glucose / \% change C - peptide )$$

$$angle = radian * 57.296$$

$$hypotenuse =$$

$$sqrt ( \% change glucose * \% change glucose ) \text{} + ( \% change C - peptide * \% change C - peptide ) )$$

(The formulas use percent change of glucose and C-peptide instead of actual values to standardize the units of the sides of the triangle.)

A Cox regression model can be developed which includes the calculated angle of change over 6 months within each quadrant as an independent variable for predicting type 1 diabetes. Because an individual's vector of change can only fall into one of the quadrants, the values of the other quadrants are designated as 0. Based on this paradigm the model is shown to be significantly predictive of type 1 diabetes. However, other models can also be predictive if the calculated angle is subtracted from 900 for certain quadrants.

The equation below shows the coefficients of the directional quadrant angles (qangles) used for an exemplary model that detects the greatest difference between the oral insulin and placebo groups (p<0.001).

$$WQE = 0.02455 * qangle 1 + 0.01464 * qangle 2 + 0.00831 * qangle 3 - 0.00465 * qangle 4$$

with qangle1=RUQ, qangle2=LUQ, qangle3=LLQ, and qangle4=RLQ (Since 3 directional quadrants of the 4 would always be negative for an individual, indicator variables coded as “0” were used.)

The summary below shows the steps for the calculation of the WQE:
Calculations of glucose and C-peptide coordinates for baseline and 6-month GCRC centroids.
Conversion of changes of glucose and C-peptide centroid coordinates into percent change.
Identify directionality quadrant for vector between baseline and 6-month coordinates.
Calculate within quadrant angle between horizontal and vector using standard formula based on right triangles.
Convert negative angles in directional quadrants 2 and 4 (qangle2 and qangle4) to positive.
   Use calculated angle for qangle1 and qangle2. Use (90°-calculated angle) for qangle3 and (90 -qangle4) in the model.

The procedures for the angle transformations from negative to positive, and the subtractions of angles from 90° are shown below:
If the vector for centroid change is in the RUQ, then qangle1 calculated angle
If the vector for centroid change is in the LUQ, then qangle2=calculated angle*(−1)
If the vector for centroid change is in the LLQ, then qangle3=90-calculated angle
If the vector for centroid change is in the LLQ, then qangle4=90-calculated angle*(−1)

In some embodiments, Ordinal Directional Endpoint (ODE) can be used. Using the 3600 scale, a value for the 4 quadrants can be assigned as indicated below according to prior evidence of sequential directionality during the longitudinal progression to type 1 diabetes.
Lower right quadrant (RLQ): 0°
Lower left quadrant (LLQ): 90°
Upper right quadrant (RUQ): 180°
Upper left quadrant (LUQ): 270°

The value can be added to the value of the qangle obtained from the WQE model. The sum is then divided by 360 to create a scale with a maximum value of 1.00.

The summary of steps for the ODE calculation can be the same as that for the WQE except that instead of inserting the qangle value into a model, the qangle is added to the designated values indicated above for the directional quadrants.

In some embodiments, high-risk factors for identification of predisposed subjects include having first or second degree relatives with diagnosed type-1 diabetes, an impaired fasting glucose level (e.g., at least one determination of a glucose level of 100-125 mg/dl after fasting (8 hours with no food)), an impaired glucose tolerance in response to a 75 g OGTT (e.g., at least one determination of a 2-hr glucose level of 140-199 mg/dl in response to a 75 g OGTT), an HbA1c between 5.7-6.4% or an increase in HbA1c of greater than or equal to 10% when compared to HbA1c values over the prior 12 month period, an HLA type of DR3, DR4 or DR7 in a Caucasian, an HLA type of DR3 or DR4 in a person of African descent, an HLA type of DR3, DR4 or DR9 in a person of Japanese descent, exposure to viruses (e.g., coxsackie B virus, enteroviruses, adenoviruses, rubella, cytomegalovirus, Epstein-Barr virus), a positive diagnosis according to art accepted criteria of at least one other autoimmune disorder (e.g., thyroid disease, celiac disease), and/or the detection of autoantibodies, particularly ICAs and type 1 diabetes-associated autoantibodies, in the serum or other tissues. In some embodiments, the subject identified as predisposed to developing type 1 diabetes has at least one of the risk factors described herein and/or as known in the art. The present disclosure also encompasses identification of subjects predisposed to development of type 1 diabetes, wherein said subject presents a combination of two or more, three or more, four or more, or more than five of the risk factors disclosed herein or known in the art.

Serum autoantibodies associated with type 1 diabetes or with a predisposition for the development of type 1 diabetes are islet-cell autoantibodies (e.g., anti-ICA512 autoantibodies), glutamic acid decarbamylase autoantibodies (e.g., anti-GAD65 autoantibodies), IA2 antibodies, ZnT8 antibodies and/or anti-insulin autoantibodies. Accordingly, in a specific example in accordance with this embodiment, the invention encompasses the treatment of an individual with detectable autoantibodies associated with a predisposition to the development of type 1 diabetes or associated with early stage type 1 diabetes (e.g., anti-IA2, anti-ICA512, anti-GAD or anti-insulin autoantibodies), wherein said individual has not been diagnosed with type 1 diabetes and/or is a first or second degree relative of a type-1 diabetic. In some embodiments, the presence of the autoantibodies is detected by ELISA, electrochemoluminescence (ECL), radioassay (see, e.g., Yu et al., 1996, J. Clin. Endocrinol. Metab. 81:4264-4267), agglutination PCR (Tsai et al, ACS Central Science 2016 2 (3), 139-147) or by any other method for immunospecific detection of antibodies described herein or as known to one of ordinary skill in the art.

β-cell function prior to, during, and after therapy may be assessed by methods described herein or by any method known to one of ordinary skill in the art. For example, the Diabetes Control and Complications Trial (DCCT) research group has established the monitoring of percentage glycosylated hemoglobin (HA1 and HA1c) as the standard for evaluation of blood glucose control (DCCT, 1993, N. Engl. J. Med. 329:977-986). Alternatively, characterization of daily insulin needs, C-peptide levels/response, hypoglycemic episodes, and/or FPIR may be used as markers of β-cell function or to establish a therapeutic index (See Keymeulen et al., 2005, N. Engl. J. Med. 352:2598-2608; Herold et al., 2005, Diabetes 54:1763-1769; U.S. Pat. Appl. Pub. No. 2004/0038867 A1; and Greenbaum et al., 2001, Diabetes 50:470-476, respectively). For example, FPIR is calculated as the sum of insulin values at 1 and 3 minutes post IGTT, which are performed according to Islet Cell Antibody Register User's Study protocols (see, e.g., Bingley et al., 1996, Diabetes 45:1720-1728 and McCulloch et al., 1993, Diabetes Care 16:911-915).

In some embodiments, the individuals predisposed to develop TlD can be a non-clinically diabetic subject who is a relative of a patient with TlD. In some embodiments, the non-clinically diabetic subject has 2 or more diabetes-related autoantibodies selected from islet cell antibodies (ICA), insulin autoantibodies (IAA), and antibodies to glutamic acid decarboxylase (GAD), tyrosine phosphatase (IA-2/ICA512) or ZnT8.

In some embodiments, the non-clinically diabetic subject has abnormal glucose tolerance on oral glucose tolerance test (OGTT). Abnormal glucose tolerance on OGTT is defined as a fasting glucose level of 110-125 mg/dL, or 2 hour plasma of >140 and <200 mg/dL, or an intervening glucose value at 30, 60, or 90 minutes on OGTT >200 mg/dL.

In some embodiments, the non-clinically diabetic subject who will respond to the anti-CD3 antibody such as teplizumab is negative for ZnT8 antibodies. In some embodiments, such non-clinically diabetic subject is HLA-DR4+. In some embodiments, such non-clinically diabetic subject is not HLA-DR3+. In some embodiments, such non-clinically diabetic subject is HLA-DR4+ and is not HLA-DR3+. In some embodiments, such non-diabetic subject is negative for antibodies against ZnT8, is HLA-DR4+ and is not HLA-DR3+. In some embodiments, such non-clinically diabetic subject who will respond to the anti-CD3 antibody such as teplizumab demonstrates an increase, following administration (e.g., after 1 month, after 2 months, after 3 months, or longer or shorter), in the frequency (or relative amount) of TIGIT+KLRG1+CD8+ T-cells (e.g., by flow cytometry) in peripheral blood mononuclear cells.

In some embodiments, the prophylactically effective amount comprises a 10 to 14-day course of subcutaneous (SC) injection or intravenous (IV) infusion of the anti-CD3 antibody such as teplizumab at 10-1100 micrograms/meter squared (μg/m²). In one example, the prophylactically effective amount comprises a 14-day course IV infusion of the anti-CD3 antibody such as teplizumab at 51 μg/m², 103 μg/m², 207 μg/m², and 413 μg/m², on days 1-4, respectively, and one dose of 826 μg/m² on each of days 5-14. In some embodiments, the prophylactically effective amount delays median time to clinical diagnosis of TlD by at least 50%, at least 80%, or at least 90%, by from about 50% to about 90%. In some embodiments, the prophylactically effective amount delays median time to clinical diagnosis of TlD by at least 12 months, at least 18 months, at least 24 months, at least 36 months, at least 48 months, or at least 60 months, or longer or by from about 12 months to about 28 months, from about 12 months to about 24 months, from about 12 months to about 36 months, from about 12 months to about 48 months, from about 12 months to about 60 months or longer.

In some embodiments, the course of dosing with the anti-CD3 antibody such as teplizumab can be repeated at 2 month, 4 month, 6 month, 8 month, 9 month, 10 month, 12 month, 15 month, 18 month, 24 month, 30 month, or 36 month intervals. In some embodiments, efficacy of the treatment with the anti-CD3 antibody such as teplizumab is determined as described herein, or as is known in the art, at 2 months, 4 months, 6 months, 9 months, 12 months, 15 months, 18 months, 24 months, 30 months, or 36 months subsequent to the previous treatment.

In some embodiments, a subject is administered one or more unit doses of approximately 0.5-50 ug/kg, approximately 0.5-40 ug/kg, approximately 0.5-30 ug/kg, approximately 0.5-20 ug/kg, approximately 0.5-15 ug/kg, approximately 0.5-10 ug/kg, approximately 0.5-5 ug/kg, approximately 1-5 ug/kg, approximately 1-10 ug/kg, approximately 20-40 ug/kg, approximately 20-30 ug/kg, approximately 22-28 ug/kg or approximately 25-26 ug/kg of the anti-CD3 antibody such as teplizumab to prevent, treat or ameliorate one or more symptoms of TlD. In some embodiments, a subject is administered one or more unit doses of about 200 ug/kg, 178 ug/kg, 180 ug/kg, 128 ug/kg, 100 ug/kg, 95 ug/kg, 90 ug/kg, 85 ug/kg, 80 ug/kg, 75 ug/kg, 70 ug/kg, 65 ug/kg, 60 ug/kg, 55 ug/kg, 50 ug/kg, 45 ug/kg, 40 ug/kg, 35 ug/kg, 30 ug/kg, 26 ug/kg, 25 ug/kg, 20 ug/kg, 15 ug/kg, 13 ug/kg, 10 ug/kg, 6.5 ug/kg, 5 ug/kg, 3.2 ug/kg, 3 ug/kg, 2.5 ug/kg, 2 ug/kg, 1.6 ug/kg, 1.5 ug/kg, 1 ug/kg, 0.5 ug/kg, 0.25 ug/kg, 0.1 ug/kg, or 0.05 ug/kg of the anti-CD3 antibody such as teplizumab to prevent, treat or ameliorate one or more symptoms of TlD.

In some embodiments, a subject is administered one or more doses of the anti-CD3 antibody such as teplizumab at about 5-1200 ug/m², for example, 51-826 ug/m². In some embodiments, a subject is administered one or more unit doses of 1200 ug/m², 1150 ug/m², 1100 ug/m², 1050 ug/m², 1000 ug/m², 950 ug/m², 900 ug/m², 850 ug/m², 800 ug/m², 750 ug/m², 700 ug/m², 650 ug/m², 600 ug/m², 550 ug/m², 500 ug/m², 450 ug/m², 400 ug/m², 350 ug/m², 300 ug/m², 250 ug/m², 200 ug/m², 150 ug/m², 100 ug/m², 50 ug/m², 40 ug/m², 30 ug/m², 20 ug/m², 15 ug/m², 10 ug/m², or 5 ug/m² of the anti-CD3 antibody such as teplizumab to prevent, treat, slow the progression of, delay the onset of or ameliorate one or more symptoms of TlD.

In some embodiments, the subject is administered a treatment regimen comprising one or more doses of a prophylactically effective amount of the anti-CD3 antibody such as teplizumab, wherein the course of treatment is administered over 2 days, 3 days, 4 days, 5 days, 6 days, 7 days, 8 days, 9 days, 10 days, 11 days, 12 days, 13 days or 14 days. In some embodiments, the treatment regimen comprises administering doses of the prophylactically effective amount every day, every 2nd day, every 3rd day or every 4th day. In some embodiments, the treatment regimen comprises administering doses of the prophylactically effective amount on Monday, Tuesday, Wednesday, Thursday of a given week and not administering doses of the prophylactically effective amount on Friday, Saturday, and Sunday of the same week until 14 doses, 13 doses, 12 doses, 11 doses, 10 doses, 9 doses, or 8 doses have been administered. In some embodiments, the dose administered is the same each day of the regimen.

In some embodiments, a subject is administered a treatment regimen comprising one or more doses of a prophylactically effective amount of the anti-CD3 antibody such as teplizumab, wherein the prophylactically effective amount is 200 ug/kg/day, 175 ug/kg/day, 150 ug/kg/day, 125 ug/kg/day, 100 ug/kg/day, 95 ug/kg/day, 90 ug/kg/day, 85 ug/kg/day, 80 ug/kg/day, 75 ug/kg/day, 70 ug/kg/day, 65 ug/kg/day, 60 ug/kg/day, 55 ug/kg/day, 50 ug/kg/day, 45 ug/kg/day, 40 ug/kg/day, 35 ug/kg/day, 30 ug/kg/day, 26 ug/kg/day, 25 ug/kg/day, 20 ug/kg/day, 15 ug/kg/day, 13 ug/kg/day, 10 ug/kg/day, 6.5 ug/kg/day, 5 ug/kg/day, 3.2 ug/kg/day, 3 ug/kg/day, 2.5 ug/kg/day, 2 ug/kg/day, 1.6 ug/kg/day, 1.5 ug/kg/day, 1 ug/kg/day, 0.5 ug/kg/day, 0.25 ug/kg/day, 0.1 ug/kg/day, or 0.05 ug/kg/day; and/or wherein the prophylactically effective amount is 1200 ug/m²/day, 1150 ug/m²/day, 1100 ug/m²/day, 1050 ug/m²/day, 1000 ug/m²/day, 950 ug/m²/day, 900 ug/m²/day, 850 ug/m²/day, 800 ug/m²/day, 750 ug/m²/day, 700 ug/m²/day, 650 ug/m²/day, 600 ug/m²/day, 550 ug/m²/day, 500 ug/m²/day, 450 ug/m²/day, 400 ug/m²/day, 350 ug/m²/day, 300 ug/m²/day, 250 ug/m² day, 200 ug/m²/day, 150 ug/m²/day, 100 ug/m²/day, 50 ug/m²/day, 40 ug/m² day, 30 ug/m²/day, 20 ug/m²/day, 15 ug/m²/day, 10 ug/m²/day, or 5 ug/m²/day.

In some embodiments, the intravenous dose of 1200 ug/m² or less, 1150 ug/m² or less, 1100 ug/m² or less, 1050 ug/m² or less, 1000 ug/m² or less, 950 ug/m² or less, 900 ug/m² or less, 850 ug/m² or less, 800 ug/m² or less, 750 ug/m² or less, 700 ug/m² or less, 650 ug/m² or less, 600 ug/m² or less, 550 ug/m² or less, 500 ug/m² or less, 450 ug/m² or less, 400 ug/m² or less, 350 ug/m² or less, 300 ug/m² or less, 250 ug/m² or less, 200 ug/m² or less, 150 ug/m² or less, 100 ug/m² or less, 50 ug/m² or less, 40 ug/m² or less, 30 ug/m² or less, 20 ug/m² or less, 15 ug/m² or less, 10 ug/m² or less, or 5 ug/m² or less of the anti-CD3 antibody such as teplizumab is administered over about 24 hours, about 22 hours, about 20 hours, about 18 hours, about 16 hours, about 14 hours, about 12 hours, about 10 hours, about 8 hours, about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat or ameliorate one or more symptoms of type 1 diabetes. In some embodiments, the total dosage over the duration of the regimen is a total of less than 9000 ug/m², 8000 ug/m², 7000 ug/m², 6000 ug/m², and may be less than 5000 ug/m², 4000 ug/m², 3000 ug/m², 2000 ug/m², or 1000 ug/m². In some embodiments, the total dosage over the duration of the regimen is a total of more than 9000 ug/m², for example from about 9000 ug/m² to about 14000 ug/m². In some embodiments, the daily dosage administered in the regimen is 100 ug/m² to 200 ug/m², 100 ug/m² to 500 ug/m², 100 ug/m² to 1000 ug/m², or 500 ug/m² to 1100 ug/m².

In some embodiments, the dose escalates over the first fourth, first half or first ⅔ of the doses (e.g., over the first 2, 3, 4, 5, or 6 days of a 10, 12, 14, 16, 18 or 20-day regimen of one dose per day) of the treatment regimen until the daily prophylactically effective amount of the anti-CD3 antibody such as teplizumab is achieved. In some embodiments, a subject is administered a treatment regimen comprising one or more doses of a prophylactically effective amount of the anti-CD3 antibody such as teplizumab, wherein the prophylactically effective amount is increased by, e.g., 0.01 ug/kg, 0.02 ug/kg, 0.04 ug/kg, 0.05 ug/kg, 0.06 ug/kg, 0.08 ug/kg, 0.1 ug/kg, 0.2 ug/kg, 0.25 ug/kg, 0.5 ug/kg, 0.75 ug/kg, 1 ug/kg, 1.5 ug/kg, 2 ug/kg, 4 ug/kg, 5 ug/kg, 10 ug/kg, 15 ug/kg, 20 ug/kg, 25 ug/kg, 30 ug/kg, 35 ug/kg, 40 ug/kg, 45 ug/kg, 50 ug/kg, 55 ug/kg, 60 ug/kg, 65 ug/kg, 70 ug/kg, 75 ug/kg, 80 ug/kg, 85 ug/kg, 90 ug/kg, 95 ug/kg, 100 ug/kg, or 125 ug/kg each day; or increased by, e.g., 1 ug/m², 5 ug/m², 10 ug/m², 15 ug/m², 20 ug/m², 30 ug/m², 40 ug/m², 50 ug/m², 60 ug/m², 70 ug/m², 80 ug/m², 90 ug/m², 100 ug/m², 150 ug/m², 200 ug/m², 250 ug/m², 300 ug/m², 350 ug/m², 400 ug/m², 450 ug/m², 500 ug/m², 550 ug/m², 600 ug/m², or 650 ug/m², each day as treatment progresses. In some embodiments, a subject is administered a treatment regimen comprising one or more doses of a prophylactically effective amount of the anti-CD3 antibody such as teplizumab, wherein the prophylactically effective amount is increased by a factor of 1.25, a factor of 1.5, a factor of 2, a factor of 2.25, a factor of 2.5, or a factor of 5 until the daily prophylactically effective amount of the anti-CD3 antibody such as teplizumab is achieved.

In some embodiments, a subject is intramuscularly administered one or more doses of a 200 ug/kg or less, preferably 175 ug/kg or less, 150 ug/kg or less, 125 ug/kg or less, 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, to prevent, treat or ameliorate one or more symptoms of TlD.

In some embodiments, a subject is subcutaneously administered one or more doses of a 200 ug/kg or less, preferably 175 ug/kg or less, 150 ug/kg or less, 125 ug/kg or less, 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, to prevent, treat or ameliorate one or more symptoms of TlD.

In some embodiments, a subject is intravenously administered one or more doses of a 100 ug/kg or less, preferably 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, to prevent, treat or ameliorate one or more symptoms of TlD. In some embodiments, the intravenous dose of 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat or ameliorate one or more symptoms of TID.

In some embodiments, a subject is orally administered one or more doses of a 100 ug/kg or less, preferably 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, to prevent, treat or ameliorate one or more symptoms of TlD. In some embodiments, the oral dose of 100 ug/kg or less, 95 ug/kg or less, 90 ug/kg or less, 85 ug/kg or less, 80 ug/kg or less, 75 ug/kg or less, 70 ug/kg or less, 65 ug/kg or less, 60 ug/kg or less, 55 ug/kg or less, 50 ug/kg or less, 45 ug/kg or less, 40 ug/kg or less, 35 ug/kg or less, 30 ug/kg or less, 25 ug/kg or less, 20 ug/kg or less, 15 ug/kg or less, 10 ug/kg or less, 5 ug/kg or less, 2.5 ug/kg or less, 2 ug/kg or less, 1.5 ug/kg or less, 1 ug/kg or less, 0.5 ug/kg or less, or 0.2 ug/kg or less of the anti-CD3 antibody such as teplizumab, otelixizumab or foralumab, is administered over about 6 hours, about 4 hours, about 2 hours, about 1.5 hours, about 1 hour, about 50 minutes, about 40 minutes, about 30 minutes, about 20 minutes, about 10 minutes, about 5 minutes, about 2 minutes, about 1 minute, about 30 seconds or about 10 seconds to prevent, treat or ameliorate one or more symptoms of TlD.

In some embodiments in which escalating doses are administered for the first days of the dosing regimen, the dose on day 1 of the regimen is 5-100 ug/m2/day, for example 51 ug/m2/day and escalates to the daily dose as recited immediately above by day 3, 4, 5, 6 or 7. For example, on day 1, the subject is administered a dose of approximately 51 ug/m²/day, on day 2 approximately 103 ug/m²/day, on day 3 approximately 207 ug/m²/day, on day 4 approximately 413 ug/m²/day and on subsequent days of the regimen (e.g., days 5-14) 826 ug/m²/day. In some embodiments, on day 1, the subject is administered a dose of approximately 227 ug/m²/day, on day 2 approximately 459 ug/m²/day, on day 3 and subsequent days, approximately 919 ug/m²/day. In some embodiments, on day 1, the subject is administered a dose of approximately 284 ug/m²/day, on day 2 approximately 574 ug/m²/day, on day 3 and subsequent days, approximately 1148 ug/m²/day.

In some embodiments, the initial dose is ¼, to ½, to equal to the daily dose at the end of the regimen but is administered in portions at intervals of 6, 8, 10 or 12 hours. For example, a 13 ug/kg/day dose is administered in four doses of 3-4 ug/kg at intervals of 6 hours to reduce the level of cytokine release caused by administration of the antibody. In some embodiments, to reduce the possibility of cytokine release and other adverse effects, the first 1, 2, 3, or 4 doses or all the doses in the regimen are administered more slowly by intravenous administration. For example, a dose of 51 ug/m²/day may be administered over about 5 minutes, about 15 minutes, about 30 minutes, about 45 minutes, about 1 hour, about 2 hours, about 4 hours, about 6 hours, about 8 hours, about 10 hours, about 12 hours, about 14 hours, about 16 hours, about 18 hours, about 20 hours, and about 22 hours. In some embodiments, the dose is administered by slow infusion over a period of, e.g., 20 to 24 hours. In some embodiments, the dose is infused in a pump, preferably increasing the concentration of antibody administered as the infusion progresses.

In some embodiments, a set fraction of the doses for the 51 ug/m²/day to 826 ug/m²/day regimen described above is administered in escalating doses. In some embodiments, the fraction is 1/10, ¼, ⅓, ½, ⅔ or ¾ of the daily doses of the regimens described above. Accordingly, when the fraction is 1/10, the daily doses will be 5.1 ug/m² on day 1, 10.3 ug/m² on day 2, 20.7 g/m² on day 3, 41.3 ug/m² on day 4, and 82.6 ug/m² on days 5 to 14. When the fraction is ¼, the doses will be 12.75 ug/m² on day 1, 25.5 ug/m² on day 2, 51 ug/m² on day 3, 103 ug/m² on day 4, and 207 ug/m² on days 5 to 14. When the fraction is ⅓, the doses will be 17 ug/m² on day 1, 34.3 ug/m² on day 2, 69 ug/m² on day 3, 137.6 ug/m² on day 4, and 275.3 ug/m² on days 5 to 14. When the fraction is ½, the doses will be 25.5 ug/m² on day 1, 51 ug/m² on day 2, 103 ug/m² on day 3, 207 ug/m² on day 4, and 413 ug/m² on days 5 to 14. When the fraction is ⅔, the doses will be 34 ug/m² on day 1, 69 ug/m² on day 2, 137.6 ug/m² on day 3, 275.3 ug/m² on day 4, and 550.1 ug/m² on days 5 to 14. When the fraction is ¾, the doses will be 38.3 ug/m² on day 1, 77.3 ug/m² on day 2, 155.3 ug/m² on day 3, 309.8 ug/m² on day 4, and 620 ug/m² on days 5 to 14. In some embodiments, the regimen is identical to one of those described above but only over days 1 to 4, days 1 to 5, or days 1 to 6. For example, in some embodiments, the doses will be 17 ug/m² on day 1, 34.3 ug/m² on day 2, 69 ug/m² on day 3, 137.6 ug/m² on day 4, and 275.3 ug/m² on days 5 and 6.

In some embodiments, the anti-CD3 antibody such as teplizumab, otelixizumab or foralumab, is not administered by daily doses over a number of days, but is rather administered by infusion in an uninterrupted manner over 4 hours, 6 hours, 8 hours, 10 hours, 12 hours, 15 hours, 18 hours, 20 hours, 24 hours, 30 hours or 36 hours. The infusion may be constant or may start out at a lower dosage for, for example, the first 1, 2, 3, 5, 6, or 8 hours of the infusion and then increase to a higher dosage thereafter. Over the course of the infusion, the patient receives a dose equal to the amount administered in the 5 to 20-day regimens set forth above. For example, a dose of approximately 150 ug/m², 200 ug/m², 250 ug/m², 500 ug/m², 750 ug/m², 1000 ug/m², 1500 ug/m², 2000 ug/m², 3000 ug/m², 4000 ug/m², 5000 ug/m², 6000 ug/m², 7000 ug/m², 8000 ug/m², 9000 ug/m², 10000 ug/m², 11000 ug/m², 12000 ug/m², 13000 ug/m², or 14000 ug/m². In particular, the speed and duration of the infusion is designed to minimize the level of free anti-CD3 antibody such as teplizumab, otelixizumab or foralumab in the subject after administration. In some embodiments, the level of free anti-CD3 antibody such as teplizumab should not exceed 200 ng/ml free antibody. In addition, the infusion is designed to achieve a combined T cell receptor coating and modulation of at least 50%, 60%, 70%, 80%, 90%, 95% or of 100%.

In some embodiments, the anti-CD3 antibody such as teplizumab, otelixizumab or foralumab is administered chronically to treat, prevent, or slow or delay the onset or progression, or ameliorate one or more symptoms of type 1 diabetes. For example, in some embodiments, a low dose of the anti-CD3 antibody such as teplizumab is administered once a month, twice a month, three times per month, once a week or even more frequently either as an alternative to the 6 to 14-day dosage regimen discussed above or after administration of such a regimen to enhance or maintain its effect. Such a low dose may be anywhere from 1 ug/m² to 100 ug/m², such as approximately 5 ug/m², 10 ug/m², 15 ug/m², 20 ug/m², 25 ug/m², 30 ug/m², 35 ug/m², 40 ug/m², 45 ug/m², or 50 ug/m².

In some embodiments, the subject may be re-dosed at some time subsequent to administration of the anti-CD3 antibody such as teplizumab, otelixizumab or foralumab dosing regimen, for example, based upon one or more physiological parameters or may be done as a matter of course. Such redosing may be administered and/or the need for such redosing evaluated 2 months, 4 months, 6 months, 8 months, 9 months, 1 year, 15 months, 18 months, 2 years, 30 months or 3 years after administration of a dosing regimen and may include administering a course of treatment every 6 months, 9 months, 1 year, 15 months, 18 months, 2 years, 30 months or 3 years indefinitely.

### EXAMPLE

### Example 1: The Deterrence of Rapid Metabolic Decline within 3 Months after Teplizumab Treatment in Individuals at High Risk for Type 1 Diabetes

### Abstract

Endpoints that provide an early identification of treatment effects are needed to implement type 1 diabetes prevention trials more efficiently. To this end, we assessed whether metabolic endpoints can be used to detect a teplizumab effect on rapid β-cell decline within 3 months after treatment in high-risk individuals in the TrialNet teplizumab trial. Glucose and C-peptide response curves (GCRCs) were constructed by plotting mean glucose and C-peptide values from 2-hour oral glucose tolerance tests on a 2-dimensional grid. Groups were compared visually for changes in GCRC shape and movement. GCRC changes reflected marked metabolic deterioration in the placebo group within 3 months of randomization. By 6 months, GCRCs resembled typical GCRCs at diagnosis. In contrast, GCRC changes in the teplizumab group suggested metabolic improvement. Quantitative comparisons, including two novel metabolic endpoints that indicate GCRC changes, the Within Quadrant Endpoint (WQE) and the Ordinal Directional Endpoint (ODE), were consistent with visual impressions of an appreciable treatment effect at 3 and 6-month timepoints. In conclusion, an analytic approach combining visual evidence with novel endpoints, demonstrated that Teplizumab delays rapid metabolic decline, and improves the metabolic state within 3 months after treatment; this effect extends for at least 6 months.

### Introduction

Type 1 diabetes is an autoimmune disease resulting in insulin deficiency due to the destruction of pancreatic beta cells (1). In the Type 1 Diabetes TrialNet TN10 Anti-CD3 prevention study, a single 14-day course of the Fc receptor-nonbinding anti-CD3$ monoclonal antibody, teplizumab, was able to delay the onset of diabetes by 32.5 months in a group of autoantibody positive, high-risk individuals (2; 3). In a follow-up longitudinal analysis of that study (3), after a decline in C-peptide responses prior to entry into the trial, teplizumab treatment improved average C-peptide area under the curve (AUC) over a period of 6 months. Also, treatment reversed an observed decline in insulin secretion before enrollment.

These findings suggested that metabolic endpoints could be of value for gaining more precise information about the effect of teplizumab. The utility of metabolic endpoints to examine the teplizumab effect could then potentially be generalized to assess preventive treatments in other clinical trials. C-peptide responses from oral glucose or mixed-meal tolerance tests (OGTTs; MMTTs) have mainly been utilized to evaluate β-cell function both before and after the diagnosis of diabetes (4). However, several studies have now shown that combined stimulated glucose and C-peptide markers improve the prediction of type 1 diabetes, the identification of heterogeneity within autoantibody positive populations, and the detection of subtle changes in R-cell function (5-11).

Thus, we have reasoned that these combined measures could be used to examine 2 important questions: (1) Among individuals at high risk for type 1 diabetes, is there a substantive teplizumab effect on β-cell function within 3 months after the 14-day course of treatment, and if so, does the effect persist for at least 6 months?(2) Is there a prospect of rapid β-cell decline among high risk individuals within 3 months if teplizumab is not administered?We used two composite glucose and C-peptide markers to address these questions, Index60 (8,9) and the C-peptide AUC/glucose AUC ratio (AUC Ratio). In addition, we used OGTT-derived glucose and C-peptide response curves (GCRCs) on 2-dimensional grids (2dgrids). This recently introduced methodology has extended the concept of combining glucose and C-peptide measures by utilizing a joint qualitative and quantitative approach. It has already facilitated studies of the metabolic natural history of type 1 diabetes (12), the metabolic heterogeneity of the disorder at diagnosis (13), and the metabolic effect of apotential preventive treatment (14). In another study, GCRCs (from MMTTs) were a basis for studying associations of metabolic changes with microRNAs after the diagnosis of type 1 diabetes (15).

The findings will show that teplizumab effectively preserves and improves β-cell function soon after treatment in individuals at high risk for type 1 diabetes, and that this action persists. They also show that although none in the placebo group had been diagnosed within 3 months after randomization, there was already marked metabolic deterioration in that period. Moreover, the findings demonstrate that using composite glucose and C-peptide endpoints in tandem with GCRCs provide qualitative and quantitative insights into the timing and magnitude of preventive treatment effects that are missed by using diagnostic endpoints alone in type 1 diabetes prevention trials.

### Research Design and Methods

### Trial Procedures and Design

The design of the phase 2, randomized, placebo-controlled, double-blinded TrialNet TN10 anti-CD3 prevention study (NCT01030861) has previously been reported in detail (2). Institutional-review-board approval was obtained at each participating site, with written informed consent and assent obtained before trial entry. Inclusion criteria were age ≥8 years at randomization, a history of a relative with type 1 diabetes, Stage 2 diabetes [positive titers for ≥two islet autoantibodies (anti-glutamic acid decarboxylase 65, micro insulin, anti-islet antigen 2, anti-zinc transporter 8, and/or islet-cell antibodies), and dysglycemia.] HbA1c levels were in the normal range [median (IQR) teplizumab: 5.2% (4.9%-5.4%); placebo: 5.3% (5.1%-5.4%)]. Participants were randomly assigned to teplizumab or saline and treated with a 14-day outpatient course administered as an IV infusion. OGTT C-peptide and glucose values were tested by Northwest Lipids Research Laboratories using the TOSOH C-peptide and Roche glucose assays. OGTTs with glucose values in the diabetic range were excluded from this analysis.

AUC values for C-peptide and glucose were calculated using the trapezoidal rule (16). C-Peptide AUC/glucose AUC ratio (AUC Ratio) values were obtained by calculating the ratio x 100. Index60 was calculated as 0.3695×(log fasting C-pep [ng/mL])+0.0165×60-min glucose (mg/dL) 0.3644×60-min C-pep (ng/mL)(17). Glucose and C-peptide response curves (GCRCs) were generated by plotting mean glucose (y-axis) and C-peptide values (x-axis) from OGTTs (30, 60, 90, and 120 minutes) on a 2dgrid.

### Analytic Components

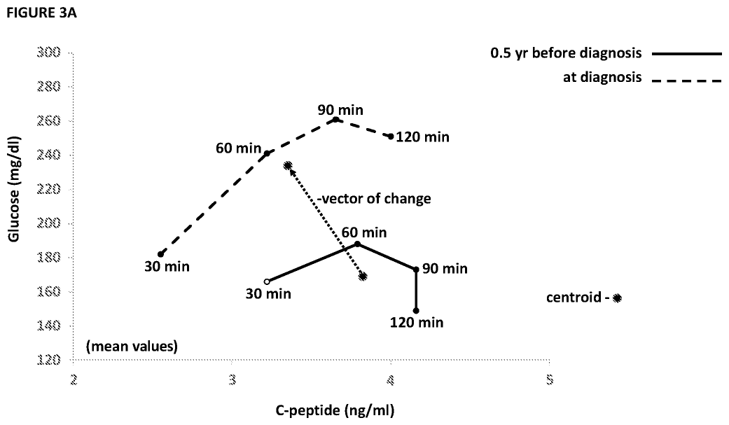

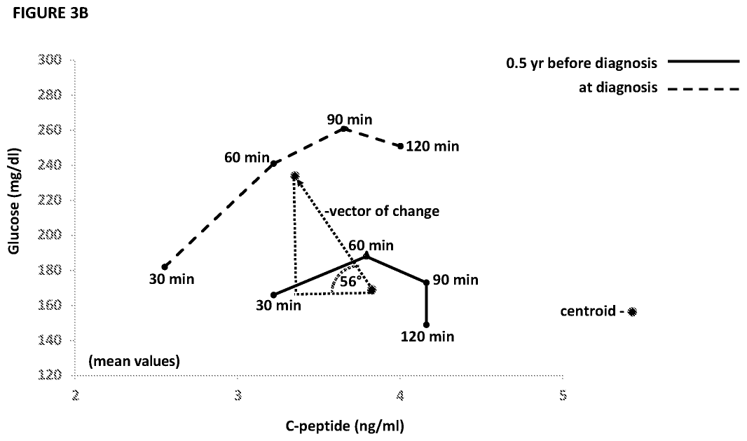

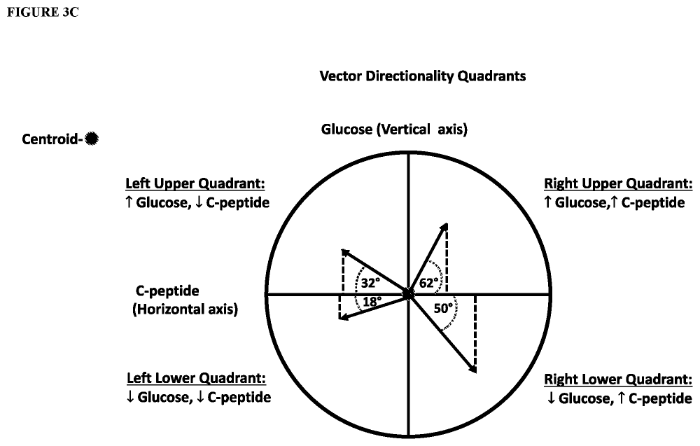

FIGS. 3A-3C includes 6 key components used in the analyses of metabolic change: 2dgrids with glucose as the y-axis and C-peptide as the x-axis, GCRCs, centroids (central point of GCRCs), vectors, directional quadrants for vectors, and angles. Shown in FIG. 3A are 2 hypothetical GCRCs from the same individuals plotted on 2dgrids from OGTT mean glucose and C-peptide values at 30, 60, 90, and 120 minutes. One of the GCRCs is typical for 6 months before diagnosis, while the other is typical for the time of diagnosis. A centroid for each GCRC is calculated (see formula). Evident are marked changes in GCRC position and shape from 6 months before diagnosis to diagnosis which are indicative of metabolic decline. The arrow is the vector for the positional change of the GCRC centroid from 6 months before diagnosis to the GCRC centroid at diagnosis. Note that the direction and magnitude of the vector are functions of the decrease in the C-peptide and the increase in the glucose from baseline to 6 months.

In FIG. 3B, a right triangle has been overlaid onto FIG. 3A. The right triangle is formed from the vector (the hypotenuse), and the changes in glucose (vertical side) and C-peptide (horizontal side). The formation of the right triangle provides a means for calculating an angle of the vector. As shown in the figure, the calculated angle for this analysis is defined as the angle between the vector (hypotenuse) and the horizontal side of the right triangle.

FIG. 3C shows a hypothetical example of vectors for each of the 4 directional quadrants: right lower quadrant; right upper quadrant; left upper quadrant; left lower quadrant. They all emanate from a baseline centroid with changes in glucose and C-peptide fixed at 0. The directional quadrant for the vector is thus dependent on whether the change of glucose and C-peptide is positive or negative. The calculated angles between the horizontal and the vector are also shown.

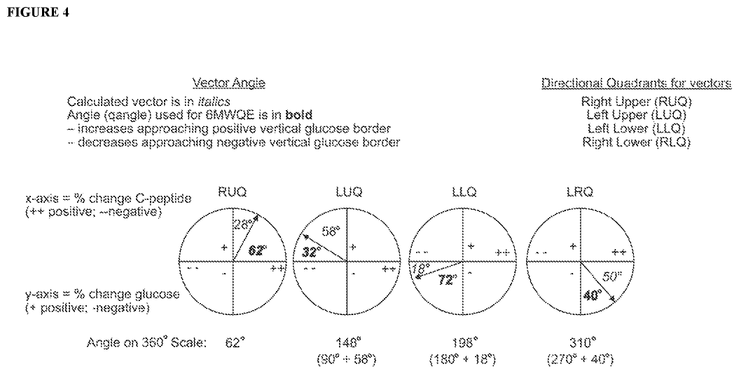

To provide quantitative comparisons that complement GCRC visual comparisons we developed two novel endpoints based on vectors and angles that indicate changes in GCRC movement over a 6-month period: the Within Quadrant Endpoint (WQE) and the Ordinal Directional Endpoint (ODE). Both endpoints utilize Cox-regression modeling that is based on GCRC movement into 4 potential quadrants reflecting changes in glucose and C-peptide. Please refer FIG. 4 for information about the development of these 2 novel endpoints.

$$Formula for Calculation of Centroid$$

$$CPEP Centroid = ( 1 / 3 ) * ( ( ( CPEP 30 + CPEP 60 ) * ( ( CPEP 30 * GLUC 60 ) - ( CPEP 60 * GLUC 30 ) ) ) + ( ( CPEP 60 + CPEP 90 ) * ( ( CPEP 60 * GLUC 90 ) - ( CPEP 90 * GLUC 60 ) ) ) + ( ( CPEP 90 + CPEP 120 ) * ( ( CPEP 90 * GLUC 120 ) - ( CPEP 120 * GLUC 90 ) ) ) + ( ( CPEP 120 + CPEP 30 ) * ( ( CPEP 120 * GLUC 30 ) - ( CPEP 30 * GLUC 120 ) ) ) ) \text{} / ( ( ( CPEP 30 * GLUC 60 ) - ( CPEP 60 * GLUC 30 ) ) + ( ( CPEP 60 * GLUC 90 ) - ( CPEP 90 * GLUC 60 ) ) + ( ( CPEP 90 * GLUC 120 ) - ( CPEP 120 * GLUC 90 ) ) + ( ( CPEP 120 * GLUC 30 ) - ( CPEP 30 * GLUC 120 ) ) )$$

$$GLUC Centroid = ( 1 / 3 ) * ( ( ( GLUC 30 + GLUC 60 ) * ( ( CPEP 30 * GLUC 60 ) - ( CPEP 60 * GLUC 30 ) ) ) + ( ( GLUC 60 + GLUC 90 ) * ( ( CPEP 60 * GLUC 90 ) - ( CPEP 90 * GLUC 60 ) ) ) + ( ( GLUC 90 + GLUC 120 ) * ( ( CPEP 90 * GLUC 120 ) - ( CPEP 120 * GLUC 90 ) ) ) + ( ( GLUG 120 + GLUC 30 ) * ( ( CPEP 120 * GLUC 30 ) - ( CPEP 30 * GLUC 120 ) ) ) ) \text{} / ( ( ( CPEP 30 * GLUC 60 ) - ( CPEP 60 * GLUC 30 ) ) + ( ( CPEP 60 * GLUC 90 ) - ( CPEP 90 * GLUC 60 ) ) + ( ( CPEP 90 * GLUC 120 ) - ( CPEP 120 * GLUC 90 ) ) + ( ( CPEP 120 * GLUC 30 ) - ( CPEP 30 * GLUC 120 ) ) )$$

Development of 6-Month within Quadrant Endpoint and 6-Month Ordinal Directional Endpoint

The sections below will describe the development of the two novel endpoints used together in the analyses and their formulations.

### Basis for Choosing Endpoints for the Analysis

Potential endpoints were first tested for their prediction of type 1 diabetes. Those predictive of type 1 diabetes were then assessed for their performance in detecting an oral insulin treatment effect in the combined DPT-1 and Trial Net oral insulin trial cohorts (n=208; no interaction between trial and treatment). Among the endpoints studied, the within quadrant endpoint (WQE) and ordinal directional endpoint (ODE) were predictive of type 1 diabetes among 281 DPT-1 participants who were in the oral insulin or parenteral insulin control groups (p<0.001 for WQE and p=0.001 for ODE unadjusted; p<0.001 for both after adjustment for AUC glucose AUC, C-peptide AUC, age and BMI). Although other endpoints were also predictive of type 1 diabetes, WQE and ODE were superior to others for detecting an oral insulin effect. Table 4 shows the comparisons between the placebo and oral insulin groups for the WQE (p<0.001) and the ODE (p=0.005) after adjustments for baseline glucose AUC, C-peptide AUC, age and BMI. Based on these findings, we utilized the WQE and ODE to statistically assess an early teplizumab effect.

### Within Quadrant Endpoint (WQE)

The full 360° continuum was poorly predictive of type 1 diabetes. Therefore, we explored the possibility that the prediction of risk could be enhanced by dividing the 3600 continuum into its 4 directional quadrants from 0° to 90°. Each directional quadrant would be considered to have its own characteristic risk that would be predictive of overall risk when included together in a model. The directional quadrants are designated as:
Right Upper Quadrant (RUQ)
Left Upper Quadrant (LUQ)
Right Lower Quadrant (RLQ)
Left Lower Quadrant (LLQ)

As shown in the FIG. 4, angles were calculated from right triangles formed according to the directionality quadrant of an individual's vector of change. Since the calculated angles were negative in LUQ and RLQ, they were transformed to positive. The percent change of the centroid glucose from baseline to 6 months on the y-axis and percent change of the centroid C-peptide from baseline to 6 months on the x-axis were used for the calculation of the angle. The hypotenuse of the triangle represents the distance from the baseline GCRC centroid from baseline to 6 months (i.e., vector for change). The formulas for the calculated angle and hypotenuse are shown below:

$$radian = arc tangent ( \% change glucose / \% change C - peptide )$$

$$angle = radian * 57.296$$

$$hypotenuse =$$

$$sqrt ( \% change glucose * \% change glucose ) \text{} + \% change C - peptide * \% change C - peptide ) )$$

(The formulas use percent change of glucose and C-peptide instead of actual values to standardize the units of the sides of the triangle.)

Using data from the control groups of the DPT-1 and TrialNet parenteral insulin and oral insulin trials (n=281), we developed a Cox regression model which included the calculated angle of change over 6 months within each quadrant as an independent variable for predicting type 1 diabetes. Because an individual's vector of change can only fall into one of the quadrants, the values of the other quadrants were designated as 0. Based on this paradigm we found that the model was significantly predictive of type 1 diabetes. However, we also found that other models could also be predictive if the calculated angle is subtracted from 90° for certain quadrants.

FIG. 4 shows how vectors, quadrants and angles were defined. It also shows hypothetically that the angles utilized for the final model included the calculated angles of percent change in the LUQ and RLQ, and the calculated angles of percent change subtracted from 90° in the LLQ and the RLQ. In addition, we observed that by adjusting model coefficients for baseline risk with the DPT-1 risk score (10) (DPTRS), the models were more predictive of type 1 diabetes. Thus, the final model coefficients are based on adjustments for the DPTRS. The equation below shows the coefficients of the directional quadrant angles (qangles) used for the model that detected the greatest difference between the oral insulin and placebo groups (p<0.001).

$$WQE = 0.02455 * qangle 1 + 0.01464 * qangle 2 + 0.00831 * qangle 3 - 0.00465 * qangle 4$$

with qangle1=RUQ, qangle2=LUQ, qangle3=LLQ, and qangle4=RLQ (Since 3 directional quadrants of the 4 would always be negative for an individual, indicator variables coded as “0” were used.)

The summary below shows the steps necessary for the calculation of the WQE:
Calculations of glucose and C-peptide coordinates for baseline and 6-month GCRC centroids.
Conversion of changes of glucose and C-peptide centroid coordinates into percent change.
Identify directionality quadrant for vector between baseline and 6-month coordinates.
Calculate within quadrant angle between horizontal and vector using standard formula based on right triangles.
Convert negative angles in directional quadrants 2 and 4 (qangle2 and qangle4) to positive.
   Use calculated angle for qangle1 and qangle2. Use (90°-calculated angle) for qangle3 and (90 -qangle4) in the model.

The procedures for the angle transformations from negative to positive, and the subtractions of angles from 900 are shown below:
If the vector for centroid change is in the RUQ, then qangle1=calculated angle
If the vector for centroid change is in the LUQ, then qangle2=calculated angle*(−1)
If the vector for centroid change is in the LLQ, then qangle3=90-calculated angle
If the vector for centroid change is in the LLQ, then qangle4=90-calculated angle*(−1)

Example for WQE: An individual's directionality of the baseline GCRC centroid to the 6-month GCRC centroid is in the LLQ with a 320 qangle. The WQE=0.00831*(32)=0.266 (0.00831 is the regression coefficient for the LLQ directional quadrant)

6-month Ordinal Directional Endpoint (ODE)

Using the 360° scale, we assigned a value for the 4 quadrants as indicated below according to prior evidence of sequential directionality during the longitudinal progression to type 1 diabetes (15).
Lower right quadrant (RLQ): 0°
Lower left quadrant (LLQ): 90°
Upper right quadrant (RUQ): 180°
Upper left quadrant (LUQ): 270°

The value was added to the value of the qangle obtained from the WQE model. The sum is then divided by 360 to create a scale with a maximum value of 1.00.

The summary of steps necessary for the ODE calculation are the same as that for the WQE except that instead of inserting the qangle value into a model, the qangle is added to the designated values indicated above for the directional quadrants.

Example for ODE: An individual's vector directionality for change from the baseline GCRC centroid to the 6-month GCRC centroid is in the LUQ at a qangle of 49°. The 6M-ODE for that individual is:

$$270 ° + 49 ° = 319 ° / 360 ° = 0.87 units$$

where 270° represents the designated quadrant value for LUQ and 49° the value of the angle within in the LUQ.

Potential endpoints were first tested for their prediction of type 1 diabetes. Those predictive of type 1 diabetes were then assessed for their performance in detecting an oral insulin treatment effect in the combined DPT-1 and Trial Net oral insulin trial cohorts (n=208; no interaction between trial and treatment). Among the endpoints studied, the within quadrant endpoint (WQE) and ordinal directional endpoint (ODE) were predictive of type 1 diabetes among 281 DPT-1 participants who were in the oral insulin or parenteral insulin control groups (p<0.001 for WQE and p=0.001 for ODE unadjusted; p<0.001 for both after adjustment for AUC glucose AUC, C-peptide AUC, age and BMI). Although other endpoints were also predictive of type 1 diabetes, WQE and ODE were superior to others for detecting an oral insulin effect. Table 4 shows the comparisons between the placebo and oral insulin groups for the WQE (p<0.001) and the ODE (p=0.005) after adjustments for baseline glucose AUC, C-peptide AUC, age and BMI. Based on these findings, we utilized the WQE and ODE to statistically assess an early teplizumab effect.

TABLE 4
Comparison between Placebo and Oral Insulin Arms of
Endpoints Utilizing Changes in Metabolic Parameters
from Baseline to 1-year Visit*
Change from Baseline Visit to 3-month visit (n = 97 for
placebo arm, n = 110 for oral insulin arm)

| | Placebo | | Oral Insulin | | Unadjusted | Adjusted |
|---|---|---|---|---|---|---|
| Endpoint | Arm | | Arm | | p-value | p-value⁺ |
| ODE | 205 | (85) | 170 | (81) | 0.003 | 0.005 |
| WQE | 0.66 | (0.66) | 0.36 | (0.50) | <0.001 | <0.001 |

⁺Adjusted for age, BMI, baseline AUC C-peptide, and AUC glucose values
*1-year visit used to assess endpoints since oral insulin peak effect was at that time point in prior study (22)

### Statistical Analyses

T-tests and chi-square tests were used to compare groups. Linear regression was used for adjustments of variables, while proportional hazards regression was used to develop models for endpoints. Sex was not included as a covariate in analyses. All analyses were performed using the statistical program SAS (version 9.4). 2-sided p-values <0.05 were considered statistically significant.

### Results

Relevant baseline characteristics of the participants are shown in Table 5. There were no significant differences between the placebo and teplizumab groups for any demographic measure, or for glucose or C-peptide levels from baseline OGTTs. Of the 76 participants, there was sufficient OGTT data to analyze 29 placebo-treated and 41 teplizumab-treated individuals at 3 months after randomization, and 24 placebo-treated and 44 teplizumab-treated individuals at 6 months after randomization. Five individuals from the placebo group were excluded from the analysis of OGTTs at 6 months due to the diagnosis of diabetes (3 individuals), or an OGTT with glucose values in the diabetic range, but not yet meeting the diagnostic criteria of 2 consecutive diabetic-range OGTTs (2 individuals).

TABLE 5
Participant Characteristics at Baseline

| | | All | | Placebo | | Teplizumab | |
|---|---|---|---|---|---|---|---|
| | | N = 76 | | N = 32 | | N = 44 | |
| Sex | | | | | | | |
| Female | | 34 | (44.74%) | 15 | (46.88%) | 19 | (43.18%) |
| Male | | 42 | (55.26%) | 17 | (53.13%) | 25 | (56.82%) |
| Race | | | | | | | |
| Asian | | 2 | (2.63%) | 2 | (6.25%) | 0 | (0.00%) |
| Hispanic | | 2 | (2.63%) | 1 | (3.13%) | 1 | (2.27%) |
| White | | 72 | (94.74%) | 29 | (90.63%) | 43 | (97.73%) |
| Age, mean (SD) | | 18.52 | (11.51) | 17.53 | (11.10) | 19.24 | (11.87) |
| BMI at baseline, | | 22.01 | (5.60) | 22.08 | (4.39) | 21.95 | (6.39) |
| mean (SD) | | | | | | | |
| Log BMI at baseline, | | 3.06 | (0.23) | 3.08 | (0.19) | 3.05 | (0.26) |
| mean (SD) | | | | | | | |
| BMI-for-age z-score, | | 0.74 | (1.23) | 0.98 | (0.75) | 0.55 | (1.47) |
| mean (SD) | | | | | | | |
| Mean Glucose AUC, | | 159.46 | (22.69) | 155.31 | (22.94) | 162.47 | (22.28) |
| mean (SD) | | | | | | | |
| Mean C-peptide AUC, | | 1.94 | (0.79) | 1.89 | (0.72) | 1.97 | (0.84) |
| mean (SD) | | | | | | | |
| Peak C-peptide, | | 2.68 | (1.09) | 2.58 | (0.99) | 2.75 | (1.16) |
| mean (SD) | | | | | | | |
| Index60, mean (SD) | | 1.95 | (0.58) | 1.85 | (0.64) | 2.02 | (0.53) |
| DPTRS, mean (SD) | | 8.17 | (1.02) | 8.10 | (1.06) | 8.22 | (1.01) |
| C-peptide AUC/Glucose | | 12.14 | (4.71) | 12.08 | (4.34) | 12.18 | (5.01) |
| AUC (×1000) | | | | | | | |
| log(C-peptide | | −4.48 | (0.36) | −4.47 | (0.34) | −4.48 | (0.38) |
| AUC/Glucose AUC) | | | | | | | |
| 30-0 min C-peptide | | 1.07 | (0.61) | 1.11 | (0.61) | 1.04 | (0.62) |
| 60-0 min C-peptide | | 1.56 | (0.80) | 1.50 | (0.69) | 1.60 | (0.87) |
| 120-60 min C-peptide | | 0.29 | (0.69) | 0.18 | (0.84) | 0.37 | (0.56) |
| 30-0 min glucose | | 70.07 | (27.63) | 69.88 | (22.47) | 70.20 | (31.10) |
| 60-0 min glucose | | 83.92 | (35.75) | 77.53 | (37.21) | 88.57 | (34.33) |
| 120-60 min glucose | | −26.66 | (36.51) | −25.81 | (38.26) | −27.27 | (35.62) |
| Glucose, mean (SD) | | | | | | | |
| 0 | minutes | 95.32 | (10.71) | 94.94 | (13.69) | 95.59 | (8.05) |
| 30 | minutes | 165.38 | (28.55) | 164.81 | (24.17) | 165.80 | (31.62) |
| 60 | minutes | 179.24 | (35.86) | 172.47 | (38.50) | 184.16 | (33.40) |
| 90 | minutes | 169.26 | (38.96) | 163.16 | (44.27) | 173.70 | (34.44) |
| 120 | minutes | 152.58 | (32.12) | 146.66 | (34.89) | 156.89 | (29.62) |
| C-peptide, mean (SD) | | | | | | | |
| 0 | minutes | 0.61 | (0.29) | 0.61 | (0.31) | 0.61 | (0.27) |
| 30 | minutes | 1.67 | (0.76) | 1.71 | (0.79) | 1.65 | (0.75) |
| 60 | minutes | 2.16 | (0.97) | 2.11 | (0.89) | 2.21 | (1.02) |
| 90 | minutes | 2.38 | (1.10) | 2.28 | (0.98) | 2.45 | (1.19) |
| 120 | minutes | 2.46 | (1.06) | 2.29 | (0.97) | 2.58 | (1.11) |

Assessments of Metabolic Responses with GCRCs 3Months and 6Months after Treatment

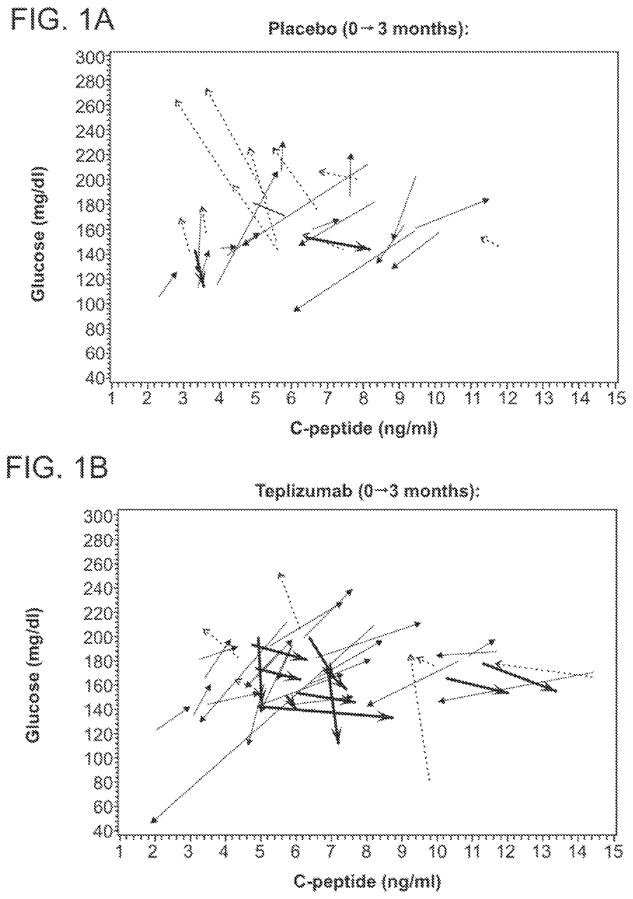

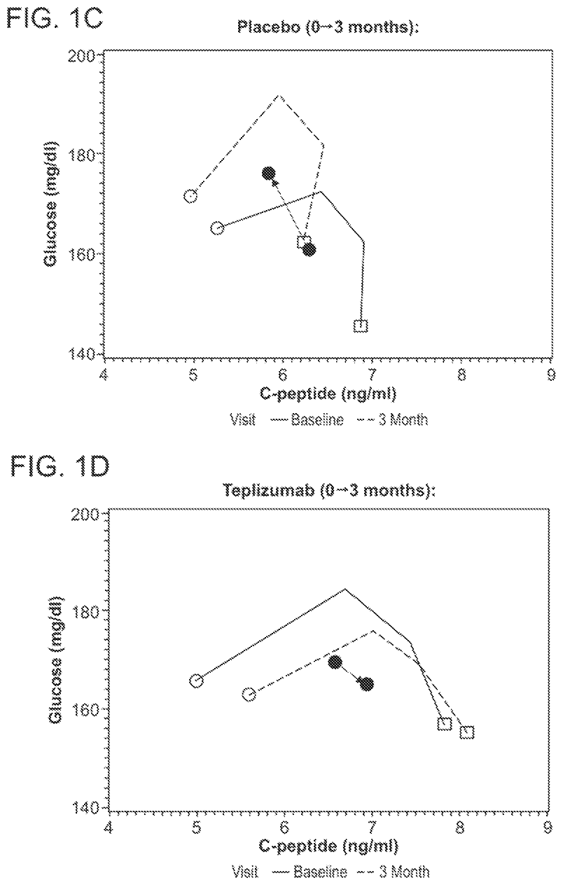

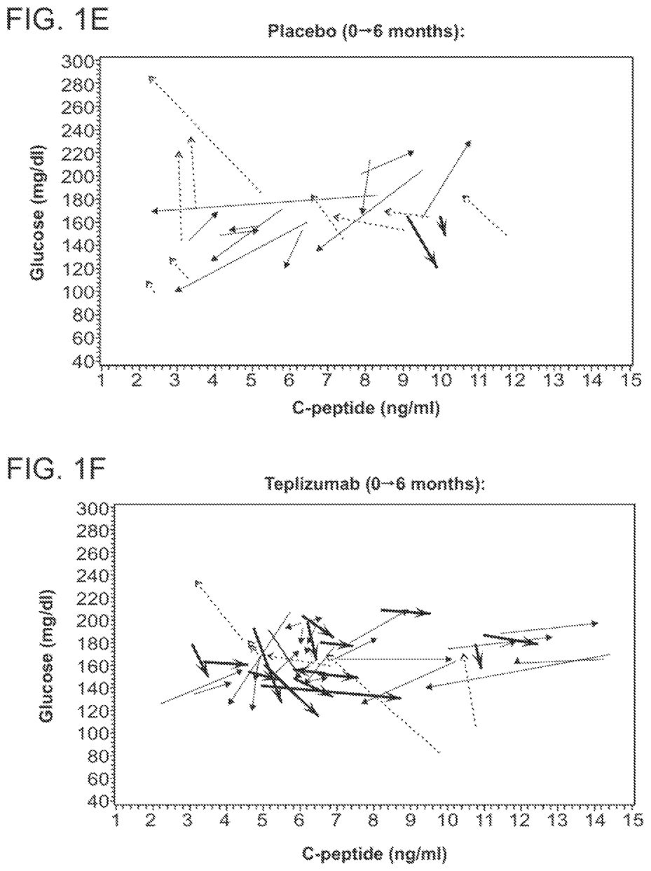

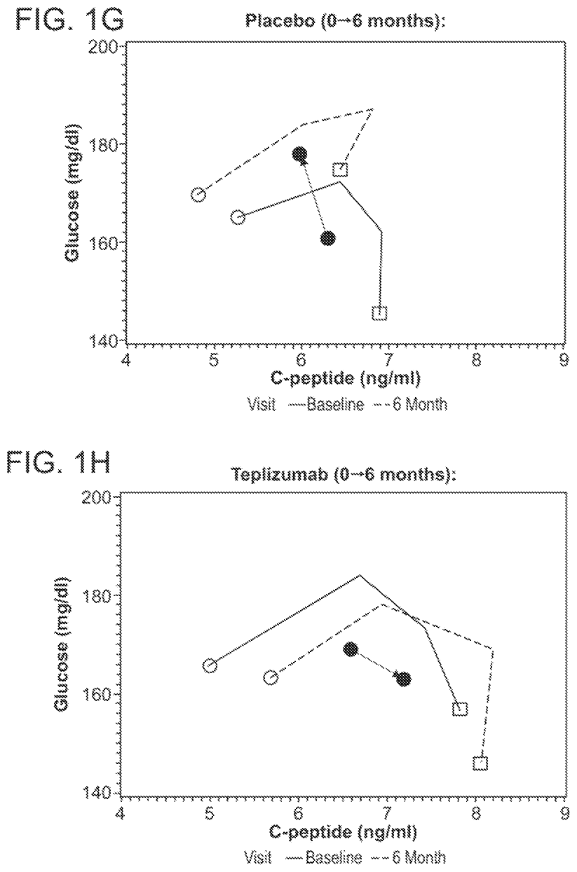

To determine whether there was early evidence of a teplizumab effect on β-cell function following treatment, we compared changes of GCRCs between the placebo and teplizumab groups from randomization to 3 months. In FIGS. 1A and 1B, a vector for centroid change from randomization to the 3-month visit is plotted for each individual in the placebo and teplizumab groups. In these vector plots, 11/29 individuals (37.9%) in the placebo group versus 6/41 (14.6%) in the teplizumab group had vectors directed toward the left upper directional quadrant (decreasing C-peptide and increasing glucose), suggesting worsening metabolic function. This contrasted with 11/41 individuals (26.8%) in the teplizumab group versus 2/29 (6.9%) in the placebo group who had vectors directed toward the right lower quadrant (increasing C-peptide and decreasing glucose), suggesting improving metabolic function. Amongst all four quadrants, the frequencies of placebo vs. teplizumab-treated participants were significantly different (overall p=0.045) (Table 1).

TABLE 1
Frequencies of individual GCRC Vectors of change according to directional quadrants
by treatment group from baseline to 3 months and baseline to 6 months

| | Baseline to 3-month visit*: | | | | Baseline to 6-month visit*: | | |
|---|---|---|---|---|---|---|---|
| | Placebo | Teplizumab | Total | | Placebo | Teplizumab | Total |
| Right Upper | 8 | 15 | 23 | Right Upper | 4 | 13 | 17 |
| Quadrant | | | | Quadrant | | | |
| Left Upper | 11 | 6 | 17 | Left Upper | 11 | 5 | 16 |
| Quadrant | | | | Quadrant | | | |
| Left Lower | 8 | 9 | 17 | Left Lower | 7 | 11 | 18 |
| Quadrant | | | | Quadrant | | | |
| Right Lower | 2 | 11 | 13 | Right Lower | 2 | 15 | 17 |
| Quadrant | | | | Quadrant | | | |
| Total | 29 | 41 | 70 | Total | 24 | 44 | 68 |

*Chi-Square test showed significantly different distributions between treatment groups at 3-month (p = 0.045) and 6-month (p = 0.004) intervals.

Depicted in FIGS. 1C-1D are GCRCs constructed according to glucose and C-peptide mean values for each OGTT time point, as well as vectors indicative of GCRC centroid changes from randomization to 3 months. Progressive metabolic dysfunction was evident in the placebo group by 3 months, with the vector for GCRC centroid change over this period directed toward the left upper quadrant (decreasing C-peptide and increasing glucose). In contrast, the vector for GCRC centroid change in the teplizumab group has nearly opposite directionality, which is toward the right lower quadrant (increasing C-peptide and decreasing glucose). The magnitude of the teplizumab effect was evident in the 1530 (out of a maximum of 180°) directional difference between the placebo vector and the teplizumab vector,

The changes in the AUC Ratio and Index60 from baseline to 3 months were consistent with the large difference in directionality of the vectors. There were significant differences in changes between the placebo and teplizumab for both (p<0.01 after adjustments for the baseline parameter, age, and BMI; Table 2). Moreover, there was evidence of improvement in the teplizumab group: the AUC Ratio increased (p<0.01) and Index60 decreased (p<0.05). (Adjustments could not be performed in the paired analyses due to multicollinearity.)

TABLE 2
Comparisons between placebo and teplizumab arms
of endpoints based upon metabolic changes from
baseline to 3 months and baseline to 6 months

| | | | | |
|---|---|---|---|---|
| | Placebo | Teplizumab | Unadjusted | Adjusted⁺ |
| Endpoint | Arm | Arm | p-value | p-value |

Change from Baseline Visit to 3-month visit (n = 29 for
placebo arm, n = 41 for teplizumab arm)

| | | | | |
|---|---|---|---|---|
| WQE | 0.70 | 0.39 | 0.026 | 0.072 |
| | (0.62) | (0.52) | | |
| ODE | 219 | 163 | 0.018 | 0.038 |
| | (92) | (97) | | |
| ΔAUC Ratio | −0.254 | 0.425 | 0.001 | 0.003 |
| | (0.80, 29) | (0.87, 41) | | |
| ΔIndex60 | 0.471 | −0.271 | <0.001 | 0.002 |
| | (0.92, 29) | (0.81, 41) | | |

Change from Baseline Visit to 6-month visit (n = 24 for
placebo arm, n = 44 for teplizumab arm)

| | | | | |
|---|---|---|---|---|
| WQE | 0.66 | 0.23 | <0.001 | <0.001 |
| | (0.47) | (0.46) | | |
| ODE | 221 | 137 | <0.001 | 0.002 |
| | (87) | (94) | | |
| ΔAUC Ratio | −0.379 | 4.159 | 0.002 | 0.005 |
| | (1.17) | (1.42) | | |
| ΔIndex60 | 0.481 | −0.159 | <0.007 | 0.021 |
| | (1.10) | (0.77) | | |

⁺ODE and WQE were adjusted for age, BMI, and for baseline AUC C-peptide and AUC glucose;
ΔAUC C-peptide/AUC Glucose and ΔIndex60 were adusted for age, BMI, and baseline Index60.

Also notable in FIGS. 1C-1D is the appreciable conformational change in the GCRC of the placebo group from randomization to 3 months. Particularly evident is the increased upward slope between 30 and 60 minutes, which is typical of changes during the progression to type 1 diabetes (12). In contrast, the slope of the teplizumab group is almost the same.

The pattern for the individual vectors at 6 months was similar to the pattern at 3 months. As shown in FIGS. 1E-1F, the directionalities of the individual vectors were leftward and upward in the placebo group, suggesting metabolic worsening, whereas they moved rightward and downward in the teplizumab group, suggesting metabolic improvement. Thus, a higher percentage of the placebo group had vectors directed to the left upper quadrant (45.8% of placebo vs. 11.4% of teplizumab, whereas a higher percentage of the teplizumab group had vectors directed towards the right lower quadrant (8.3% of placebo vs. 34.1% of teplizumab). Differences were also evident in the vectors for centroid movement of the GCRCs derived from OGTT mean values (FIGS. 1G-1H). The directional difference between the placebo and teplizumab vectors from randomization to 6 months was 138°. The difference in the vector frequencies between the groups amongst all four quadrants was significant (overall p=0.004) (Table 1).

Differences in changes of the AUC Ratio and Index60 between the groups were again significant, but less so (p=0.005 and p=0.021, respectively, after adjustments; Table 2). The increase in the AUC Ratio was also again evident within the teplizumab group from baseline to 6 months (p<0.01), but Index60 did not decrease significantly.

In FIGS. 1G-1H, it is evident that by 6 months after randomization the shape of the GCRC has become substantially more pathological such that it has assumed a shape resembling the characteristic GCRC shape at diagnosis (12). Specifically, the placebo GCRC from 30 to 90 minutes has become almost linear, attributable to a slope from 60 to 90 minutes that has become less downward. The teplizumab GCRC shape changed to a much lesser degree.

Quantitative Assessments of Treatment Effects Using Vector Angles within Directional Quadrants

The analyses above showed obvious differences in GCRC vectors between the placebo and teplizumab groups in the teplizumab trial within 3 months and at least to 6 months after the 14-day treatment. These differences were corroborated by the differences between the groups in Index60 and the AUC Ratio. However, those composite glucose and C-peptide measures were not based on the directionality of vectors indicative of GCRC change. Thus, we strived to develop quantitative endpoints that would be more directly indicative of differences in vectors between placebo and treatment groups. Two such endpoints were developed: the within quadrant endpoint (WQE) and ordinal directional endpoint (ODE) (see Research Design and Methods). Both endpoints were derived from Diabetes Prevention Trial-Type 1 changes in GCRC centroid locations at 6-month intervals, which was the shortest interval between OGTTs that was available for analysis.

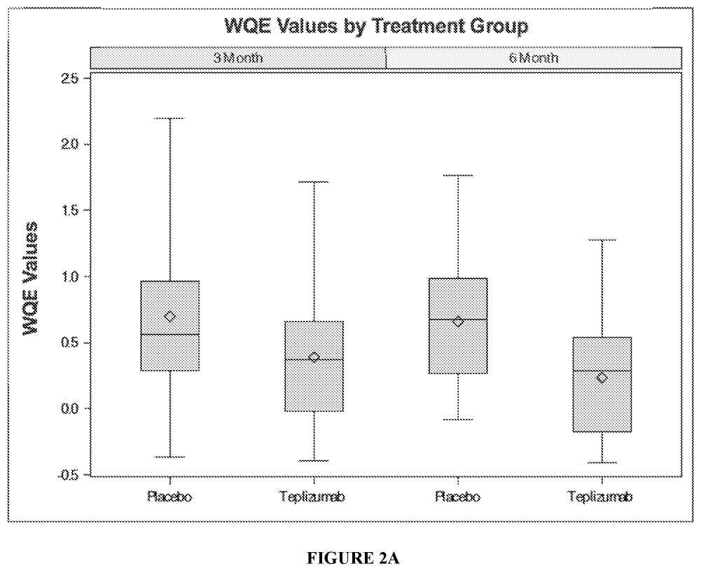

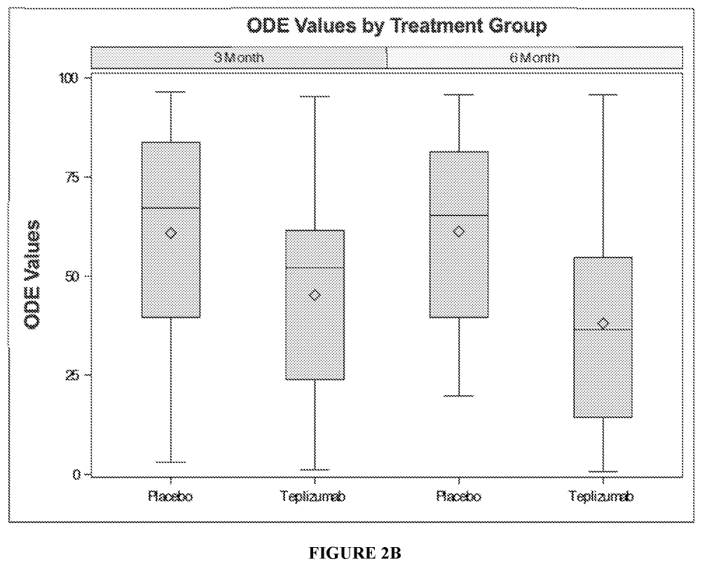

The visual difference in movement of the GCRCs from baseline to 3 months between the placebo and treatment groups was confirmed statistically with ODE and WQE (FIGS. 2A and 2B). ODE values were significantly lower for the teplizumab group (p<0.05 after adjustments). WQE values were also significantly lower in the teplizumab group before adjustments (p<0.05), but they only trended towards a difference after adjustments (p=0.072).

WQE and ODE differences between placebo and teplizumab groups at 6 months were appreciably greater than at 3 months: WQE was significantly lower for the teplizumab group (p<0.01) as was the ODE (p<0.01). Table 2, which summarizes the differences between the placebo and teplizumab groups according to the endpoints, shows that whereas Index60 and the AUC Ratio differed more between groups at 3 months, WQE and ODE differed more at 6 months.

### Conclusions

The teplizumab trial showed that a single course of treatment could delay the onset of type 1 diabetes (2). However, that trial and other prevention trials utilizing the standard endpoint of time-to-diagnosis have been lengthy. Also, although teplizumab was very effective in delaying type 1 diabetes, information has been lacking about the timing of its effect and its impact on the metabolic state. We thus used existing and new metabolic markers to serve as endpoints in order to gain this information. Such early readouts could not only be helpful for understanding the effects of preventive treatments on R-cell function, but also possibly lead to a shortening of prevention trials.

The measurement of the C-peptide AUC in response to oral glucose before diagnosis or to a mixed meal after diagnosis is often used to assess insulin responsiveness; however, it is not necessarily the most sensitive measure for identifying changes in R-cell function. Multiple studies have suggested that C-peptide loss in type 1 diabetes may be best presented and understood in the context of changes in glucose (5-11). We have used a new approach in this study which melds qualitative and quantitative information to better understand insulin secretory dynamics in response to glucose. Using this approach, the findings provided strong evidence that teplizumab deters severe β-cell decline, and possibly improves function, by 3 months after treatment in a high-risk population. Moreover, an effect persists for at least 6 months after treatment. These findings have important implications, since they indicate that even at an advanced stage of metabolic progression prior to diagnosis, the effect of teplizumab is rapid enough to be highly impactful.

GCRCs and their centroids, together with vectors and their angles, had major roles in the analysis. As was evident from the GCRC changes on the 2dgrid, the use of those elements resulted in a visual contrast between the failing metabolic state of the placebo group and the apparent improvement of the teplizumab group. Those elements were also used as a basis for developing the new endpoints, WQE and ODE, which were used in quantitative comparisons of changes between the placebo and teplizumab groups. Together with the AUC Ratio and Index60, those endpoints confirmed the visual impressions that teplizumab deterred rapid metabolic decline in high risk individuals soon after the 14-day treatment at baseline. Table 3 summarizes how methodology based on GCRC movement and shape enhanced our understanding of the timing, magnitude, and potential clinical importance of the teplizumab effect.

TABLE 3
The list below shows the substantive contributions to
the analysis from using GCRCs plotted on 2dgrids.

The rapid pathologic evolution of placebo group GCRCs, evident both in
changes of grid location and shape of the placebo group, highlighted the
crucial importance of the early treatment effect of teplizumab.
The GCRCs of the teplizumab group had minimal conformational change
with a vector directionality indicative of improvement. This enhanced
the quantitative evidence which suggested that teplizumab administration
could reverse pathologic changes.
The striking, almost opposite, difference in directionality (153°) between
the placebo vector and the teplizumab vector demonstrated the strength
of the teplizumab effect, further reinforcing the value of the medication
in high-risk individuals.
At 6 months after randomization, the teplizumab effect was most
apparent using quantitative GCRC-derived endpoints (WQE and ODE);
these corroborated visual impressions. Although based on 6-month
OGTTs, complementary quantitative information for GCRCs was also
provided by those endpoints at 3 months.
Tracking individual responses with vectors provided a granular
way of assessing responsiveness to teplizumab, both
qualitatively and quantitatively.

The findings from these analyses highlight advantages of utilizing vectors in addition to scalars as metabolic endpoints for analyses of treatment effects. Prior analyses of preventive measures have been dependent on scalars, which only provide information about magnitude, whereas vectors provide information about direction in addition to magnitude. The vectors of the GCRC movements on the 2dgrids clearly added information that could not have been ascertained from scalar endpoints alone.

We had previously utilized vectors of GCRC change as evidence of an oral insulin effect for preserving β-cell function in a post-hoc analysis among individuals at high risk for type 1 diabetes in the DPT-1 and TrialNet oral insulin trials (14). Interestingly, the vectors in the present teplizumab analysis were much further apart between the placebo and treatment groups than in the oral insulin analysis. Whereas the difference in vector direction between the placebo and teplizumab groups in the teplizumab trial spanned a difference from the left upper directional quadrant (placebo) to the right lower directional quadrant (teplizumab), the difference between the placebo and oral insulin vectors was mostly confined to the right upper directional quadrant. The greater vector separation in the teplizumab trial could be based on differences in therapeutic mechanisms between teplizumab and oral insulin and/or differences in the magnitude of effect. It appears that analyses of preventive treatment effects would be incomplete if vectors are ignored, and there is only reliance on scalars.

The detection of treatment effects by WQE and ODE endpoints might not generalize to trials at other stages of disease, since those in the teplizumab trial were selected for high risk. Also, it is quite possible that other endpoints relating to GCRC centroids and their vectors for change will be found that are superior to WQE and ODE. Nevertheless, the performance of those endpoints in the analyses validates visual impressions derived from GCRCs, suggesting that a combined qualitative and quantitative use of vectors to examine metabolic change can be a valuable approach for assessing effects of preventive treatments.

Of note, this is the also first report to utilize changes in Index60 as an endpoint for demonstrating a metabolic effect in an analysis of a T1D prevention study. In the aggregate, these and prior results (14) suggest that multiple metabolic endpoints, including Index60, the AUC ratio, and WQE and ODE, which combine changes in glucose and C-peptide, can provide a readout of treatment effects in type 1 diabetes prevention studies. Subtle variability in treatment group differences detected between the oral insulin and teplizumab trials could reflect differences in study populations or therapeutic interventions. The change from baseline to 3 months in Index60 and AUC ratio differed more between the placebo and teplizumab groups than WQE and ODE, whereas the opposite was evident from baseline to 6 months. Since the development of both WQE and ODE was based upon 6-month data, it is not surprising that they performed better from baseline to 6 months. Although the analysis of the teplizumab study suggested that Index60 and AUC ratio could possibly be more sensitive for detecting an early effect, this might not generalize to other trials. The choice of specific metabolic endpoints for prevention trials will depend upon factors that could impact their relevance and performance such as the target population, objectives, interventions, and trial designs. An advantage of WQE and ODE over other measures is that they directly complement the cogent visual impressions derived from GCRCs (outlined in Table 3).

The 0 to 30-minute interval is not included in GCRCs, since the magnitude of changes in metabolic measures from 0 to 30 minutes is much greater than the magnitude at each of the other time intervals. Its incorporation would thus minimize the 30 to 120-minute visualization. Likewise, from a quantitative perspective, the 0 to 30-minute interval would carry an inordinate weight for calculations of centroids and vectors, which would further complicate analyses. Although 0 and 30-minute C-peptide and glucose measures are not included in GCRCs, we did adjust for baseline AUC C-peptide and AUC glucose values in the analyses. Now that quantitative measures have been developed to complement the visualization of GCRCs, along with their centroids and vectors, an important future direction will be to study the influence of changes from 0 to 30 minutes upon changes in form and location of GCRCs.

The applicability of short-term, interim metabolic endpoints for prevention trials will need further study. Primary metabolic endpoints have been used successfully in new-onset type 1 diabetes trials, usually with a defined follow-up of 1 year, to assess treatment efficacy for delaying the further loss of insulin secretion. Positive findings of metabolic outcomes in these new-onset trials have been used to justify treatments in pre-diagnosis prevention trials (18-21). Our findings suggest that information from short-term prevention trials prior to diagnosis could also be used for deciding whether larger pre-diagnostic prevention trials are warranted for a particular treatment.

Short-term metabolic endpoints are likely to have several other utilities for evaluating pre-diagnosis preventive treatments. They could be used for stopping rules in trials if prespecified effects are not reached. One novel approach for using a metabolic endpoint would be using it in combination with the diagnosis endpoint. It is even conceivable that metabolic endpoints could serve as primary endpoints for certain trials. These endpoints could also provide important pharmacologic information from trials, such as the timing of treatment effects, as was evident in the present study.

The prior longitudinal study of teplizumab (3) examined the average effect of teplizumab treatment on C-peptide AUC over a 6-month period. That study did not use GCRC methodology and composite glucose and C-peptide endpoints to assess the specific timing of a teplizumab effect, nor show the importance of getting ahead of the impending severe loss of 0-cell functionality with the use of teplizumab among high risk individuals. As previously mentioned, the analysis of the oral insulin trials (14) utilized GCRC vectors, but to a much lesser extent.

This study had some limitations. Three individuals from the placebo group had already developed diabetes at the 6-month timepoint, and so were excluded from the 6-month analysis. This not only decreased the sample size, but likely led to the appearance of less severe metabolic decline in the placebo group. Such bias would attenuate a teplizumab effect at 6 months rather than enhance it. Since all teplizumab trial participants were required to be positive for multiple islet autoantibodies with metabolic abnormalities, future analyses will be needed to determine if our findings are applicable to at-risk populations with less severe baseline disease, such as individuals testing positive for a single islet autoantibody.

GCRC vectors in right lower and left upper quadrants appear to reflect improving or worsening metabolic status, whereas vectors in the right upper and left lower quadrants could reflect more intermediate changes consistent with changes in insulin secretory kinetics or insulin sensitivity. However, gold standard measures of beta cell function, such as glucose-potentiated arginine clamps, would be required to truly define these changes. Quantitative treatment group comparisons (Table 2) were adjusted for BMI and age, but due to the severity of beta cell dysfunction in this population, we chose not to adjust for OGTT-based modeled measures of insulin resistance (22).

WQE and ODE were both significantly associated with diabetes progression in DPT-1; however, the detection of a treatment effect by a metabolic endpoint is not purely a function of its ability to predict type 1 diabetes. For example, an individual may have an improved C-peptide response to a treatment, yet still go on to develop diabetes, while conversely, another individual might not exhibit improved C-peptide secretion to treatment, yet not develop type 1 diabetes during follow-up. Thus, a critical future direction is an analysis of relationships between the ability of endpoints to detect a treatment effect vs. ability to predict diabetes.

The development of the WQE and ODE endpoints involved some complexity, but their applications for studies would mainly be based on standard formulas for calculations of angles. Thus, while we have included explanations to describe the development of this novel methodology in the current manuscript, practically, implementation of these measurements would simply involve utilization of a formula, similar to other accepted indices, such as Index60 (17).

In conclusion, an analysis of glucose and C-peptide changes based upon changes of GCRCs and their centroids on 2dgrids provided visual evidence of a teplizumab treatment effect that was early enough to deter a rapid decline in R-cell function, and possibly even improve function, in a population at high risk for type 1 diabetes. This was corroborated statistically by composite glucose and C-peptide endpoints, and by novel endpoints derived from vectors of GCRC change. These findings suggest that changes in GCRC grid location and shape, along with corresponding quantitative measures of directionality, should be integrated into analyses of type 1 diabetes prevention trials. Moreover, they add to the growing evidence that endpoints which combine both glucose and C-peptide are basic to our understanding of treatment effects in prevention trials. Future directions will involve refining applications of these endpoints to facilitate assessments of preventive treatments in both smaller and larger trials.

Modifications and variations of the described methods and compositions of the present disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure are intended and understood by those skilled in the relevant field in which this disclosure resides to be within the scope of the disclosure as represented by the following claims.

### INCORPORATION BY REFERENCE

All patents and publications mentioned in this specification are herein incorporated by reference to the same extent as if each independent patent and publication was specifically and individually indicated to be incorporated by reference.

### REFERENCES

1. DiMeglio L A, Evans-Molina C, Oram R A: Type 1 diabetes. Lancet 2018; 391:2449-2462
2. Herold K C, Bundy B N, Long S A, Bluestone J A, DiMeglio L A, Dufort M J, Gitelman S E, Gottlieb P A, Krischer J P, Linsley P S, Marks J B, Moore W, Moran A, Rodriguez H, Russell W E, Schatz D, Skyler J S, Tsalikian E, Wherrett D K, Ziegler A G, Greenbaum C J, Type 1 Diabetes TrialNet Study G: An Anti-CD3 Antibody, Teplizumab, in Relatives at Risk for Type 1 Diabetes. N Engl J Med 2019; 381:603-613
3. Sims E K, Bundy B N, Stier K, Serti E, Lim N, Long S A, Geyer S M, Moran A, Greenbaum C J, Evans-Molina C, Herold K C, Type 1 Diabetes TrialNet Study G: Teplizumab improves and stabilizes beta cell function in antibody-positive high-risk individuals. Sci Transl Med 2021; 13
4. Palmer J P, Fleming G A, Greenbaum C J, Herold K C, Jansa L D, Kolb H, Lachin J M, Polonsky K S, Pozzilli P, Skyler J S, Steffes M W: C-peptide is the appropriate outcome measure for type 1 diabetes clinical trials to preserve beta-cell function: report of an ADA workshop, 21-22 Oct. 2001. Diabetes 2004; 53:250-264
5. Sosenko J M, Krischer J P, Palmer J P, Mahon J, Cowie C, Greenbaum C J, Cuthbertson D, Lachin J M, Skyler J S, Diabetes Prevention Trial-Type 1 Study G: A risk score for type 1 diabetes derived from autoantibody-positive participants in the diabetes prevention trial-type 1. Diabetes Care 2008; 31:528-533
6. Sosenko J M, Skyler J S, Mahon J, Krischer J P, Beam C A, Boulware D C, Greenbaum C J, Rafkin L E, Cowie C, Cuthbertson D, Palmer J P, Type 1 Diabetes T, Diabetes Prevention Trial-Type 1 Study G: Validation of the Diabetes Prevention Trial-Type 1 Risk Score in the TrialNet Natural History Study. Diabetes Care 2011; 34:1785-1787
7. Sosenko J M, Skyler J S, Mahon J, Krischer J P, Beam C A, Boulware D C, Greenbaum C J, Rafkin L E, Cowie C, Cuthbertson D, Palmer J P, Type 1 Diabetes TrialNet Study G, Diabetes Prevention Trial-Type 1 Study G: The application of the diabetes prevention trial-type 1 risk score for identifying a preclinical state of type 1 diabetes. Diabetes Care 2012; 35:1552-1555
8. Nathan B M, Boulware D, Geyer S, Atkinson M A, Colman P, Goland R, Russell W, Wentworth J M, Wilson D M, Evans-Molina C, Wherrett D, Skyler J S, Moran A, Sosenko J M, Type 1 Diabetes T, Diabetes Prevention Trial-Type 1 Study G: Dysglycemia and Index60 as Prediagnostic End Points for Type 1 Diabetes Prevention Trials. Diabetes Care 2017; 40:1494-1499
9. Sosenko J M, Skyler J S, DiMeglio L A, Beam C A, Krischer J P, Greenbaum C J, Boulware D, Rafkin L E, Matheson D, Herold K C, Mahon J, Palmer J P, Type 1 Diabetes TrialNet Study G, Diabetes Prevention Trial-Type 1 Study G: A new approach for diagnosing type 1 diabetes in autoantibody-positive individuals based on prediction and natural history. Diabetes Care 2015; 38:271-276
10. Sosenko J M, Skyler J S, Mahon J, Krischer J P, Greenbaum C J, Rafkin L E, Beam C A, Boulware D C, Matheson D, Cuthbertson D, Herold K C, Eisenbarth G, Palmer J P, Type 1 Diabetes T, Diabetes Prevention Trial-Type 1 Study G: Use of the Diabetes Prevention Trial-Type 1 Risk Score (DPTRS) for improving the accuracy of the risk classification of type 1 diabetes. Diabetes Care 2014; 37:979-984
11. Ferrannini E, Mari A, Nofrate V, Sosenko J M, Skyler J S: Progression to diabetes in relatives of type 1 diabetic patients: mechanisms and mode of onset. Diabetes 2010; 59:679-685
12. Ismail H M, Cleves M A, Xu P, Libman I M, Becker D J, Marks J B, Skyler J S, Palmer J P, Sosenko J M, Type 1 Diabetes TrialNet Study G: The Pathological Evolution of Glucose Response Curves During the Progression to Type 1 Diabetes in the TrialNet Pathway to Prevention Study. Diabetes Care 2020; 43:2668-2674
13. Redondo M J, Nathan B M, Jacobsen L M, Sims E, Bocchino L E, Pugliese A, Schatz D A, Atkinson M A, Skyler J, Palmer J, Geyer S, Sosenko J M, Type 1 diabetes TrialNet Study G: Index60 as an additional diagnostic criterion for type 1 diabetes. Diabetologia 2021; 64:836-844
14. Sosenko J M, Skyler J S, Herold K C, Schatz D A, Haller M J, Pugliese A, Cleves M, Geyer S, Rafkin L E, Matheson D, Palmer J P, Type 1 Diabetes TrialNet Study G: Slowed Metabolic Decline After 1 Year of Oral Insulin Treatment Among Individuals at High Risk for Type 1 Diabetes in the Diabetes Prevention Trial-Type 1 (DPT-1) and TrialNet Oral Insulin Prevention Trials. Diabetes 2020; 69:1827-1832
15. Snowhite I, Pastori R, Sosenko J, Messinger Cayetano S, Pugliese A: Baseline Assessment of Circulating MicroRNAs Near Diagnosis of Type 1 Diabetes Predicts Future Stimulated Insulin Secretion. Diabetes 2021; 70:638-651
16. Allison D B, Paultre F, Maggio C, Mezzitis N, Pi-Sunyer F X: The use of areas under curves in diabetes research. Diabetes Care 1995; 18:245-250
17. Redondo M J, Nathan B M, Jacobsen L M, Sims E, Bocchino L E, Pugliese A, Schatz D A, Atkinson M A, Skyler J, Palmer J, Geyer S, Sosenko J M, Type 1 diabetes TrialNet Study G: Index60 as an additional diagnostic criterion for type 1 diabetes. Diabetologia 2021;
18. Hagopian W, Ferry R J, Jr., Sherry N, Carlin D, Bonvini E, Johnson S, Stein K E, Koenig S, Daifotis A G, Herold K C, Ludvigsson J, Protege Trial I: Teplizumab preserves C-peptide in recent-onset type 1 diabetes: two-year results from the randomized, placebo-controlled Protege trial. Diabetes 2013; 62:3901-3908
19. Herold K C, Hagopian W, Auger J A, Poumian-Ruiz E, Taylor L, Donaldson D, Gitelman S E, Harlan D M, Xu D, Zivin R A, Bluestone J A: Anti-CD3 monoclonal antibody in new-onset type 1 diabetes mellitus. N Engl J Med 2002; 346:1692-1698.
20. Keymeulen B, Vandemeulebroucke E, Ziegler A G, Mathieu C, Kaufman L, Hale G, Gorus F, Goldman M, Walter M, Candon S, Schandene L, Crenier L, De Block C, Seigneurin J M, De Pauw P, Pierard D, Weets I, Rebello P, Bird P, Berrie E, Frewin M, Waldmann H, Bach J F, Pipeleers D, Chatenoud L: Insulin needs after CD3-antibody therapy in new-onset type 1 diabetes. N Engl J Med 2005; 352:2598-2608
21. Sherry N, Hagopian W, Ludvigsson J, Jain S M, Wahlen J, Ferry R J, Jr., Bode B, Aronoff S, Holland C, Carlin D, King K L, Wilder R L, Pillemer S, Bonvini E, Johnson S, Stein K E, Koenig S, Herold K C, Daifotis A G, Protege Trial I: Teplizumab for treatment of type 1 diabetes (Protege study): 1-year results from a randomised, placebo-controlled trial. Lancet 2011; 378:487-497
22. Wallace T M, Levy J C, Matthews D R: Use and abuse of HOMA modeling. Diabetes Care 2004; 27:1487-1495
23. Helminen 0, Aspholm S, Pokka T, et al. HbA1c Predicts Time to Diagnosis of Type 1 Diabetes in Children at Risk. Diabetes. 2015; 64(5):1719-1727.
24. Vehik K, Boulware D, Killian M, et al. Rising hemoglobin A1C in the nondiabetic range predicts progression of type 1 diabetes as well as oral glucose tolerance tests. Diabetes Care. 2022.

1. A method of prognosing or assessing responsiveness of a therapeutic or prophylactic agent for treating or preventing type 1 diabetes (T1D), comprising:
administering the therapeutic or prophylactic agent to a subject in need thereof, and
determining a glucose and C-peptide response curve (GCRC) vector of change by plotting change over a period of time of mean glucose values and mean C-peptide values from oral glucose tolerance tests (OGTTs) on a 2-dimensional grid, wherein a directionality of the vector of change towards increasing C-peptide and decreasing glucose is indicative of metabolic improvement.

2. The method of claim 1, wherein the OGTTs comprise 1-hour, 2-hour, or 4-hour OGTT.

3. The method of claim 1, further comprising calculating Within Quadrant Endpoint (WQE) and Ordinal Directional Endpoint (ODE) from the GCRC.

4. The method of claim 1, wherein the therapeutic or prophylactic agent comprises an immunotherapeutic agent, optionally wherein the immunotherapeutic agent comprises an anti-CD3 antibody or antigen-binding fragment thereof.

5. (canceled)

6. The method of claim 4, wherein the anti-CD3 antibody is teplizumab, otelixizumab or foralumab.

7. The method of claim 4, comprising administering to the subject in need thereof a 10 to 14 day course of daily subcutaneous (SC) injection or intravenous (IV) infusion of the anti-CD3 antibody at 10-1100 micrograms/meter squared (μg/m²).

8. The method of claim 4, comprising administering to the subject in need thereof a 10 to 14 day course of the anti-CD3 antibody at a total dose of about 9000 μg/m² to about 14000 μg/m².

9. The method of claim 4, comprising administering to the subject in need thereof a 14-day course of IV infusion of the anti-CD3 antibody at 51 μg/m², 103 μg/m², 207 μg/m², and 413 μg/m², on days 1-4, respectively, and one dose of 826 μg/m² on each of days 5-14.

10. (canceled)

11. The method of claim 6, wherein the anti-CD3 antibody is teplizumab.

12. The method of claim 1, wherein the subject has stage 1, 2, 3, or 4 TlD, optionally wherein the subject has stage 1 or 2 TlD and the agent is a prophylactic agent for preventing or delaying the onset of stage 3 TlD.

13. (canceled)

14. A method of prognosing or assessing responsiveness of an anti-CD3 antibody in preventing or delaying the onset of type 1 diabetes (T1D), comprising:
administering a prophylactically effective amount of the anti-CD3 antibody to a non-clinically diabetic subject who is at risk for clinical TlD; and
determining a glucose and C-peptide response curve (GCRC) vector of change by plotting change over a period of time of mean glucose values and mean C-peptide values from oral glucose tolerance tests (OGTTs) on a 2-dimensional grid, wherein a directionality of the vector of change towards increasing C-peptide and decreasing glucose is indicative of metabolic improvement.

15. The method of claim 14, further comprising calculating Within Quadrant Endpoint (WQE) and Ordinal Directional Endpoint (ODE) from the GCRC.

16. The method of claim 14, wherein the non-clinically diabetic subject is a relative of a patient with clinical TlD, optionally wherein the non-clinically diabetic subject has two or more diabetes-related autoantibodies selected from islet cell antibodies (ICA), insulin autoantibodies (IAA), glutamic acid decarboxylase (GAD) antibodies, tyrosine phosphatase (IA-2/ICA512) antibodies, and zinc transporter 8 (ZnT8) antibodies.

17. (canceled)

18. The method of claim 14, wherein the non-clinically diabetic subject (1) is negative for zinc transporter 8 (ZnT8) antibodies, (2) is HLA-DR4+, and/or (3) is not HLA-DR3+, optionally wherein the non-clinically diabetic subject is negative for ZnT8 antibodies, is HLA-DR4+, and is not HLA-DR3+.

19-20. (canceled)

21. The method of claim 14, wherein the non-clinically diabetic subject has abnormal glucose tolerance on OGTT, optionally wherein the abnormal glucose tolerance on OGTT is a fasting plasma glucose level of 110-125 mg/dL, a 2-hour plasma glucose level of ≥140 and <200 mg/dL, or an intervening plasma glucose level of >200 mg/dL at 30, 60, 90 minutes on OGTT.

22. (canceled)

23. The method of claim 14, comprising administering to the subject in need thereof a 10 to 14 day course of subcutaneous (SC) injection or intravenous (IV) infusion of the anti-CD3 antibody at 10-1100 micrograms/meter squared (g/m²).

24. The method of claim 14, comprising administering to the subject in need thereof a 10 to 14 day course of the anti-CD3 antibody at a total dose of about 9000 μg/m² to about 14000 μg/m².

25. The method of claim 14, comprising administering to the subject in need thereof a 14-day course of IV infusion of the anti-CD3 antibody at 51 μg/m², 103 μg/m², 207 μg/m², and 413 μg/m², on days 1-4, respectively, and one dose of 826 μg/m² on each of days 5-14.

26. The method of claim 14, wherein the anti-CD3 antibody delays median time to clinical diagnosis of TlD by from about 50% to about 90% and/or by from about 12 months to about 60 months.

27. (canceled)

28. The method of claim 14, wherein the anti-CD3 antibody is teplizumab, otelixizumab or foralumab, optionally wherein the anti-CD3 antibody is teplizumab.

29. (canceled)
